(19) United States
(12) Patent Application Publication
(10) Pub. No.: US 2024/0001061 A1
(43) Pub. Date: Jan. 4, 2024

(54) FLOW REGULATION MECHANISM FOR COMPARTMENTALIZED LUNG VENTILATION

(71) Applicant: THE REGENTS OF THE UNIVERSITY OF CALIFORNIA, Oakland, CA (US)

(72) Inventors: Igor Barjaktarevic, Oakland, CA (US); Glen Meyerowitz, Sherman Oaks, CA (US)

(21) Appl. No.: 18/252,440

(22) PCT Filed: Nov. 10, 2021
(86) PCT No.: PCT/US21/58789

§ 371 Date: May 10, 2023

Related U.S. Application Data
(60) Provisional application No. 63/111,940, filed on Nov. 10, 2020.

(51) Int. Cl. A61M 16/04; A61M 16/00; A61M 16/20

(52) U.S. Cl. CPC A61M 16/0404; A61M 16/0003; A61M 16/024; A61M 16/201; A61M 2205/073

(57) ABSTRACT

A device is used to improve the standard of care for patients with physiologic or pathologic differences between regions of the lungs. Regional variations can occur between the left and right lungs, upper and lower lungs, different lobes in the lungs, or diffuse variation that does not follow a strict pattern. The device includes a method to quantify the differences between regions of the lungs. Quantification of regional variations in lung pathophysiology can be performed with a sensor, such as a pressure or gas concentration sensor, imaging scans, or alternative technology. This quantified parameter is used to regulate the gas flow to each lung region. One embodiment does this with a novel flow regulation mechanism for a double lumen endotracheal tube for compartmentalized lung ventilation where either the flow or pressure to the left and right lung can be varied with a flow regulation mechanism. The device has embedded software that is able to analyze the clinical parameters of the left and right lung in real-time, including advanced algorithms to provide improved clinical feedback to clinicians to allow them to provide the highest level of personalized and precision care to a patient.

### CROSS-REFERENCE TO RELATED APPLICATIONS

This application claims priority to U.S. Provisional Patent Application No. 63/111,940 filed Nov. 10, 2020, the contents of which are incorporated by reference herein in its entirety.

### BACKGROUND OF THE INVENTION

Lung protective mechanical ventilation strategies have been the basis of adequate management of acute respiratory distress syndrome (ARDS), a syndrome affecting 200,000 patients annually in the United States which is characterized by mortality reaching over 50% in severe cases. This conservative intervention, which has been shown to lead to absolute reduction of mortality by 9%, is guided by parameters obtained exclusively at the outer side of a ventilatory circuit in the ventilator itself, thus limiting the ability of the treating physician to account for heterogeneity in the compliance and recruitability of the two lungs.

While several methods have been proposed to diagnose heterogeneity of lung injury distribution in ARDS, there have been no adequate therapeutic approaches to remedy this problem other than patient position change with pronation. Mechanical ventilation is a method used to provide treatment to patients with ARDS.

Mechanical ventilation (MV) is a form of life support. A mechanical ventilator is a machine that takes over the work of breathing when a person is not able to breathe enough on their own. Mechanical ventilation can be noninvasive, in cases of milder degree of respiratory insufficiency, or invasive, requiring endotracheal intubation. Endotracheal intubation is a procedure by which an endotracheal tube (ETT) is inserted through the mouth into the trachea, securing the delivery of air to the lung via invasive mechanical ventilation.

In addition to serving as the conduit for delivery of mechanical breaths, the ETT protects the airway, allows for suctioning of secretions, and facilitates select procedures, including bronchoscopy. Standard single-lumen ETTs are positioned in the distal trachea and are able to distribute the air to the lungs as a single system. The distribution of the volume of delivered gas depends on physiologic and pathologic parameters, including compliance and resistance in each lung and its segments.

Being used as a conduit to enable oxygenation and ventilation between the patient lungs and the ventilator, an ETT has certain limitations: it delivers oxygen and other gases at the level of the distal trachea allowing for distribution to be subdued to the resistance of the airways of the right and left lungs and compliance of different areas of lungs. Similarly, being a conduit for exhaled air from the lungs, it does not allow for differentiation of the ventilation characteristics between the two lungs. Since the pressures in the lungs are measured at the level of the ventilator at the end of a ventilatory circuit, extra-thoracic variables such as ETT occlusion, compromise of the ventilatory circuit outside of a patient, or air leaks from the breathing circuit may lead to inaccuracies when capturing intrathoracic pressures.

Double lumen tubes (DLT) have been routinely used in cardiothoracic surgery for a single lung isolation and one-lung ventilation of the lung on a nonoperative side. These tubes comprise two individual tubes that are bonded together to allow each tube to ventilate a specific lung. The longer lumen (bronchial lumen) is designed to reach the main stem bronchus while the shorter lumen (tracheal) ends in the distal trachea. The double lumen tube can be left-sided or right-sided depending on the main stem bronchus which its distal (longer) lumen is designed to fit in.

Double lumen tubes play a crucial role in airway management during thoracic surgery, anatomical lung separation, isolating a normal lung from a diseased lung in situations such as massive hemorrhage from one lung, whole lung lavage in patients with pulmonary alveolar proteinosis, or avoidance of spillage of purulent secretions from one lung to another. Practically speaking, a double lumen tube is used to allow for single-lung ventilation by isolating the other lung. These tubes have rarely been used to offer fine monitoring of the lung ventilation or to go beyond qualitative approaches allowing or stopping the ventilation to the lung. In addition to airway rupture from traumatic placement as one of many possible complications of double lumen tube, malposition and displacement of these tubes can lead to life-threatening consequences of their use.

Thus, there is a need in the art for a device that allows for simultaneous ventilation of lungs with settings customized to physiologic requirement of each of the lungs separately. Such system requires both the ability to deliver the gas to each of the lungs based on desired settings, as well as to offer a feedback information about each lung's function and mechanics in order to guide ventilatory setup.

### SUMMARY OF THE INVENTION

A compartmentalized lung ventilation system is described. The system includes a double lumen endotracheal tube having a proximal end and a distal end, the proximal end being connected to a mechanical ventilator circuit and the distal end being positionable in the lungs of a subject, a balloon positioned within a distal portion of each lumen of the double lumen endotracheal tube, a sensor positioned within a distal portion of a wall of each lumen of the double lumen endotracheal tube, wherein each sensor is positioned distal to the balloon, a monitor communicatively connected to the embedded sensors, and a flow regulator positioned at the proximal end of the double lumen endotracheal tube. In one embodiment, the sensor is selected from the group consisting of a pressure sensor and an EtCO₂ sensor.

In one embodiment, the lung ventilation system includes a software platform comprising a regulation control module (RCM), a clinical parameter module (CPM) and an alarm module (AM), wherein at least one of the RCM, CPM and AM is programmed to regulate flow through one or both lumens of the endotracheal tube based on a signal received from the sensors. In one embodiment, the system is capable of quantifying the physiologic or pathologic differences between the left and the right lungs of a patient and adjusting the gas flow based on optimal ventilation reflected in end-tidal CO₂ (EtCO₂) and targeted pressures to the left and right lungs independently. In one embodiment, the physiologic or pathologic differences between the lungs are established based on quantifiable data collected by the sensors and appropriate baseline methods to compare with known standards of care. In one embodiment, the sensors for quantifying physiologic or pathologic values either within the patient trachea or bronchus, or directly downstream of the flow regulation mechanism outside of the subject's body. In one embodiment, the flow to the left and right lungs is regulated via at least one of the group selected from an inflatable wall portion of the lumen, an internal flapper mechanism whereby the angle of the flapper changes the effective flow area of the lumen, an external mechanism that pinches the tube wall, and a valve mechanism to provide a variable flow coefficient to the tube.

In one embodiment, the physiologic or pathologic data comprises plateau pressures, lung pressures, or EtCO₂ levels. In one embodiment, the system includes custom sensor ports, or pneumatic channels, within the double lumen endotracheal tube to allow for fluid parameters, such as pressure or gas concentration, to be detected by a sensor located outside of the patient body and the sense port will allow the sensor to measure a parameter of the fluid at a physical location of interest. In one embodiment, the pressure regulator receives feedback from the sensors, and the pressure regulator independently controls volume and pressure to the balloon or an inflatable wall portion of the lumen as a way to actively change the flow area inside the tube. In one embodiment, the system determines at least one of the group consisting of dynamic pressures in each of the two lungs, static pressures in each of the lungs, and EtCO₂ in each lung. In one embodiment, the system is used in a variety of setting including, but not limited to an in-patient setting where a patient is mechanically ventilated and intubated. In one embodiment, the system further includes tubing adapters per ISO 5356-1 to connect to standard breathing circuit tubing used for respirators and mechanical ventilators. In one embodiment, the system further includes advanced algorithms, such as a machine learning algorithm based on supervised learning, in order to use the data collected by the sensors to provide data informed care and improve patient outcomes. In one embodiment, the system provides predictive care outcomes and personalized medicine for individual patients based on the sensor data. In one embodiment, the system is trained in the following, non-inclusive, ways: supervised machine learning, where the data from the device is compared to traditional lung performance test results and other clinical tests; and unsupervised machine learning, where time-based data from the device is used to develop a model based on how future lung performance is impacted by past lung performance.

Further, a device is described that is capable of quantifying the physiologic or pathologic differences between the left and the right lungs of a patient and adjusting the gas flow based on optimal ventilation reflected in EtCO₂ and targeted pressures to the left and right lungs independently. A method to quantify the physiologic or pathologic differences between the left and right lungs of a patient using either: a pressure sensor; or a gas concentration sensor, such as EtCO₂ or end-tidal oxygen (EtO₂) whereby the physiologic or pathologic differences between the lungs are established based on the quantifiable data collected by a method indicated above and appropriate baseline methods to compare with known standards of care. The method may be located inside the airway. Embodiments of this invention place the sensors for quantifying physiologic or pathologic values either within the patient trachea or bronchus, or directly downstream of the flow regulation mechanism outside of the patient body. A mechanism to regulate flow to the left and right lungs based on quantifiable data consisting of: an inflatable wall portion of the lumen; or an internal flapper mechanism whereby the angle of the flapper changes the effective flow area of the lumen; or an external mechanism that pinches the tube wall; or a valve mechanism to provide a variable flow coefficient to the tube.

The device may be a novel double lumen tube which contains sensors, including, but not limited to pressure, gas concentration, or flow, that collect physiologic or pathologic data, such as plateau pressures, lung pressures, EtCO₂ levels, or other values from either within the respiratory tract of a patient, such as the bronchus or trachea within the patient's body. The method may include custom sensor ports, or pneumatic channels, within a novel double lumen tube. These sense ports allow for fluid parameters, such as pressure or gas concentration, to be detected by a sensor located outside of the patient body and the sense port will allow the sensor to measure a parameter of the fluid at a physical location of interest. The mechanism may include a pressure regulator that receives feedback from the sensors. The pressure regulator independently controls volume and pressure to the inflatable wall portion of the lumen as a way to actively change the flow area inside the tube. The sensor and device may be used for determination of the following clinical values: dynamic pressures in each of the two lungs; or static pressures in each of the lungs; or EtCO₂ in each lung. The device can be used in a variety of setting including, but not limited to an in-patient setting where a patient is mechanically ventilated and intubated.

The device may include tubing adapters per ISO 5356-1 to connect to standard breathing circuit tubing used for respirators and mechanical ventilators. The device may include advanced algorithms, such as a machine learning algorithm based on supervised learning, in order to use the data collected by the sensor suite to provide data-informed care and improve patient outcomes. The method and mechanism can be used to provide predictive care outcomes and personalized medicine for individual patients based on the sensor data. The method can be trained in the following, non-inclusive, ways: supervised machine learning, where the data from the device is compared to traditional lung performance test results and other clinical tests; and unsupervised machine learning, where time-based data from the device is used to develop a model based on how future lung performance is impacted by past lung performance.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts a perspective view of an exemplary compartmentalized lung ventilation device of the present invention.

FIG. 2 depicts a section view of an exemplary compartmentalized lung ventilation device of the present invention with at least one flow regulator having an inflatable member in a deflated state.

FIG. 3 depicts a section view of an exemplary compartmentalized lung ventilation device of the present invention with at least one flow regulator having an inflatable member in an inflated state.

FIG. 4 depicts a rendering of an exemplary compartmentalized lung ventilation device of the present invention with an external control module.

FIG. 5 depicts one exemplary configuration of an electromechanical control unit for at least one flow regulator, wherein the restriction of gas flow to lung 1 is being increased.

FIG. 6 depicts one exemplary configuration of an electromechanical control unit for at least one flow regulator, wherein the restriction of gas flow to lung 2 is being increased.

FIG. 7 depicts a block diagram of finite state machine for the present invention

FIG. 8 depicts one exemplary configuration of an electromechanical control unit for at least one flow regulator, wherein the restriction of gas flow to both lung 1 and lung 2 is being increased.

FIG. 9, comprising FIG. 9A through FIG. 9B, depicts one exemplary configuration of an electromechanical control unit for at least one flow regulator. FIG. 9A depicts one exemplary configuration of an electromechanical control unit for at least one flow regulator, wherein the restriction to gas flow to both lung 1 and lung 2 is decreased. FIG. 9B depicts one exemplary configuration of an electromechanical control unit for at least one flow regulator, wherein gas flow is configured to decrease the restriction from both lung 1 and lung 2.

FIG. 10 depicts a functional block diagram comprising a regulation control module (RCM), a clinical parameter module (CPM) and an alarm module (AM).

FIG. 11 depicts a functional block diagram outlining the software modules used in the device of the present invention.

FIG. 12 depicts a functional block diagram of the Finite State Machine (FSM) Transition Module of the present invention.

FIG. 13 depicts a functional block diagram of the Adjust Target Pressure Module of the present invention.

FIG. 14 depicts a functional block diagram of the Proportional-Integral-Derivative (PID) Controller Module of the present invention.

FIG. 15 depicts a functional block diagram of the Achieve Target Pressure Module of the present invention.

FIG. 16 depicts a functional block diagram of the Observational Finite State Machine (FSM) Module of the present invention.

FIG. 17 depicts a functional block diagram of the Sensors Module of the present invention.

FIG. 18 depicts a system view of an exemplary compartmentalized lung ventilation device of the present invention.

FIG. 19 depicts a system view of an exemplary compartmentalized lung ventilation device of the present invention.

FIG. 20 depicts a system view of an exemplary compartmentalized lung ventilation device of the present invention.

FIG. 21, comprising FIG. 21A through FIG. 21B, depicts a cross-sectional view of an exemplary double lumen endotracheal tube for a compartmentalized lung ventilation device of the present invention. FIG. 21A depicts a cross-sectional view of the second lumen or third lumen with an inflatable member inside the wall in a deflated state. FIG. 21B depicts a cross-sectional view of the second lumen or third lumen with an inflatable member inside the wall in an inflated state. In this configuration the inflatable member is partially inflated. The internal diameter of the lumen has been partially constricted by inflating the wall of the lumen. The shaded area of the internal diameter is a reduction in effective cross-sectional area of the internal flow area for the lumen.

FIG. 22 depicts an isometric view of the regulation mechanism inside of the first lumen or second lumen with an inflatable member inside the wall in an inflated state.

FIG. 23 depicts a section view of an exemplary double lumen endotracheal tube of the present invention.

FIG. 24 depicts a magnified view of an exemplary double lumen endotracheal tube of the present invention.

FIG. 25 is a flowchart depicting an exemplary method of regulating flow to each lung of a patient using the device of present invention

FIG. 26 depicts an exemplary benchtop device of the present invention.

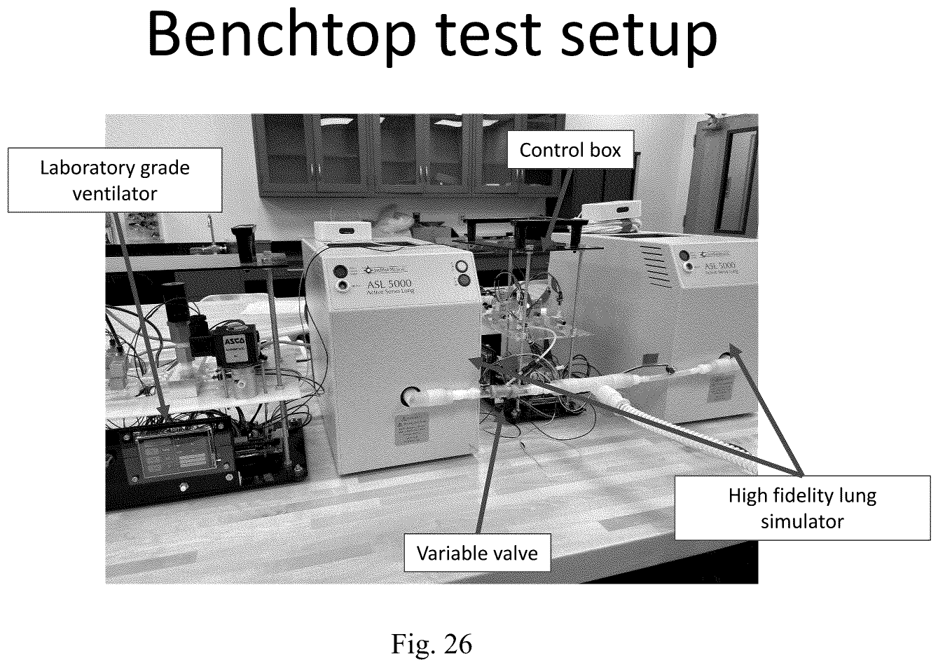

FIG. 27 depicts test matrix—test lung parameters for an exemplary experiment setup of the present invention.

FIG. 28 depicts the result of an experiment testing lung volume and pressure in a asymmetric lung model with and without the device of present invention with a compliance ratio of 10:50 (0.2).

FIG. 29 depicts the result of an experiment testing lung volume and pressure in a asymmetric lung model with and without the device of present invention with a compliance ratio of 20:50 (0.4).

FIG. 30 depicts the result of an experiment testing lung volume and pressure in a asymmetric lung model with and without the device of present invention with a compliance ratio of 30:50 (0.6).

FIG. 31 depicts the result of an experiment testing lung volume and pressure in a asymmetric lung model with and without the device of present invention with a compliance ratio of 40:50 (0.8).

FIG. 32 depicts the result of an experiment testing lung volume and pressure in a asymmetric lung model with and without the device of present invention with a compliance ratio of 50:50 (1.0).

FIG. 33 depicts the ratio of peak pressure between lung 1 and lung 2. The results demonstrate that the device of present invention corrects for the impacts of asymmetric lung injury by matching pressures between two lungs and decreasing driving pressure of mechanical ventilation.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of lung ventilation systems. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, 1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

### Flow Regulation Mechanism for Compartmentalized Lung Ventilation

The present invention relates to a device that allows for compartmentalized lung ventilation in a patient with unilateral, asymmetric, or heterogenous lung injury requiring mechanical ventilation. The device regulates gas exchange in the lungs via a double lumen tube capable of delivering variable flow rates of gas to the left and right lungs independently. The regulation of flow is adjustable by the operator and is based on quantified parameters of physiologic or pathologic differences between the left and right lung represented by parameters such as dynamic and static pressures and end-tidal carbon dioxide (EtCO₂) monitoring in each of the two lungs. This monitoring is continuous and independent in each side of the lungs and is enabled by micro-sensors located at the distal end of an adapter or implanted in each lumen of the endotracheal tube.

The invention for precise compartmentalized lung ventilation allows for direct monitoring of pressures, EtCO₂, or other physiologic or pathologic parameters in each lung independently and allows for asymmetric ventilation to left and right lung based on these parameters.

This invention enables direct intrathoracic monitoring of functions of left and right lungs simultaneously and independently, and enables the adjustment of mechanical ventilation settings tailored to pathophysiological needs of each lung.

The long-standing but heretofore unfulfilled need for a flow regulation mechanism as part of a double lumen endotracheal tube or to be used in conjunction with a double lumen endotracheal tube for compartmentalized lung ventilation which is a device capable of regulating gas by controlling either flow or pressure to the left and right lungs independently, based on data which identifies physiologic or pathologic differences between the two lungs is now met by the invention as shown and described herein.

The present invention includes a device that is able to regulate gas flow to either the left of the right lung based on physiologic or pathologic differences between the lungs. The device includes a method for observing and quantifying the differences between the lungs, which may be based on pressure sensors, gas concentration sensors, such as EtCO₂ sensors, imaging scans of the lungs, or other data. This data is parameterized in order to control the gas regulating mechanism and thus provide gas to the left and right lungs of the patient in an optimized manner to reduce lung injury and improve patient care.

In one example, an embodiment makes use of a novel double lumen tube which has pressure ports for measuring the pressure at the distal (endotracheal) tip of the bronchial and tracheal tubes which are able to provide real-time data on physiologic or pathologic changes to the patient. Internal to the wall of each lumen is a mechanism to regulate the flow of gas which serves to independently alter the flow area of the lumen and thus control the flow and pressure that is provided to either lung.

In one example, an embodiment makes use of an adapter which connects to existing double lumen tubes. This adapter contains distal sensors able to provide real-time data on pressure or EtCO₂ levels in the airway to the left and right lungs of a patient. Based on these measured values, adapter will have a mechanism internal to the lumen of the adapter to regulate the effective cross-sectional area of the lumen, thus controlling the flow of gas that is provided to either lung.

In another example, an embodiment makes use of an electropneumatic controller with embedded software which uses the pressure sensor data from each lung to determine the desired flow regulation inside the lumen. The electropneumatic controller has a closed loop algorithm that implements a proportional-integral-derivative controller (PID controller) for achieving the correct flow rate of gas through either lumen based on the distal parameters of the lungs.

In another example, an embodiment of the device has a flow regulator placed proximally and sensors placed at the distal end of the tube, and an alternative embodiment has both pressure and EtCO₂ sensors and a flow regulator placed on the proximal end of the invention.

In another example, an embodiment includes advanced algorithms that may be implemented in order to determine the impact of factors such as delivered volume, respiratory rate, gas transfer rates, airway pressures achieved during inspiration and expiration, and other factors which can provide real-time clinical feedback to clinicians to allow them to provide the best care to a patient. A method to employ this involves development of a machine learning framework that can provide predictive analytics to a clinician and improve patient care.

In short, recognizing the need to improve the ability to identify the heterogeneity of the lung disease which leads to different recruitability and variable effect of parameters such as positive end-expiratory pressure (PEEP) and offer an intervention that allows for asymmetric ventilation based on the information about lung mechanics, this invention is a flow regulation mechanism for a double lumen endotracheal tube for precise compartmentalized lung ventilation which allows for direct monitoring of pressures and EtCO₂ in each lung independently and allows for asymmetric ventilation to left and right lung based on these parameters. The invention enables direct intrathoracic monitoring of functions of left and right lungs simultaneously and independently and enables the adjustment of mechanical ventilation settings tailored to pathophysiological needs of each lung.

As previously explained, clinical management of patients with respiratory issues is generally limited due to the inability to provide appropriate care to the left and right lungs independently as they often have different physiologic or pathologic parameters. This novel technology is a device that provides the ability to regulate gas exchange to the lungs in an independent manner and enables quantification of the pathophysiological differences between the two lungs, allowing for improved patient care and reduced injury.

Many different methods exist to quantify the physiologic or pathologic changes between the lungs, such as using a pressure sensor, gas concentration sensor, imaging scans of the lungs such as x-ray or computed tomography (CT) scan, or other methods. In another example, an embodiment uses pressure sensors connected to the sense ports on the invention to measure real-time lung pressure in the left and right lungs. The device may include an EtCO₂ sensor to measure hemodynamic response of the lungs and changes in alveolar recruitment. The device may include other methods for quantifying lung changes.

As described herein, there are multiple embodiments of the present invention and are unique at least in the following ways: Regulation of the airflow for ventilation distribution: One embodiment of the device includes an inflatable portion of the wall of each lumen as a way to regulate flow in the tube. Another embodiment of the device includes an external pinch mechanism to regulate the flow through the tube. The device may include other mechanisms for regulating the flow or pressure through the tube. Quantification of physiologic or pathologic differences between the left and right lungs for regulation control: One embodiment uses sensors such as pressure or EtCO₂ feedback to provide this data. One embodiment uses advanced software algorithms to drive data-informed patient care based on data collected by the device. These algorithms may make use of supervised machine learning algorithms where the data for the device performance and clinical guidance is informed by previous clinical uses of this device and also from datasets where the device performance is compared with long term patient trends.

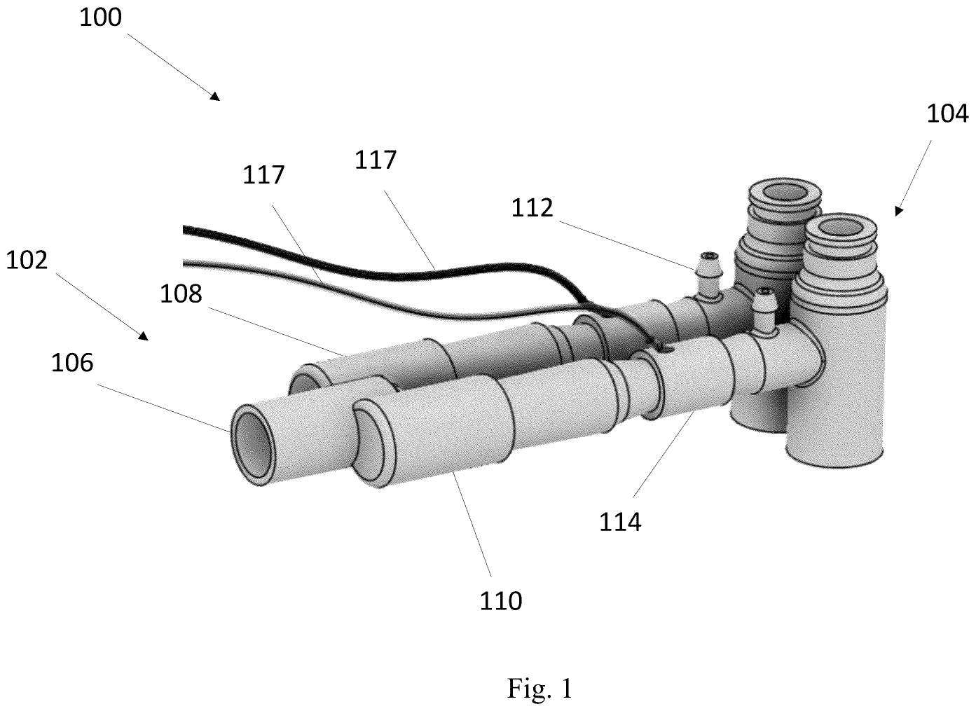

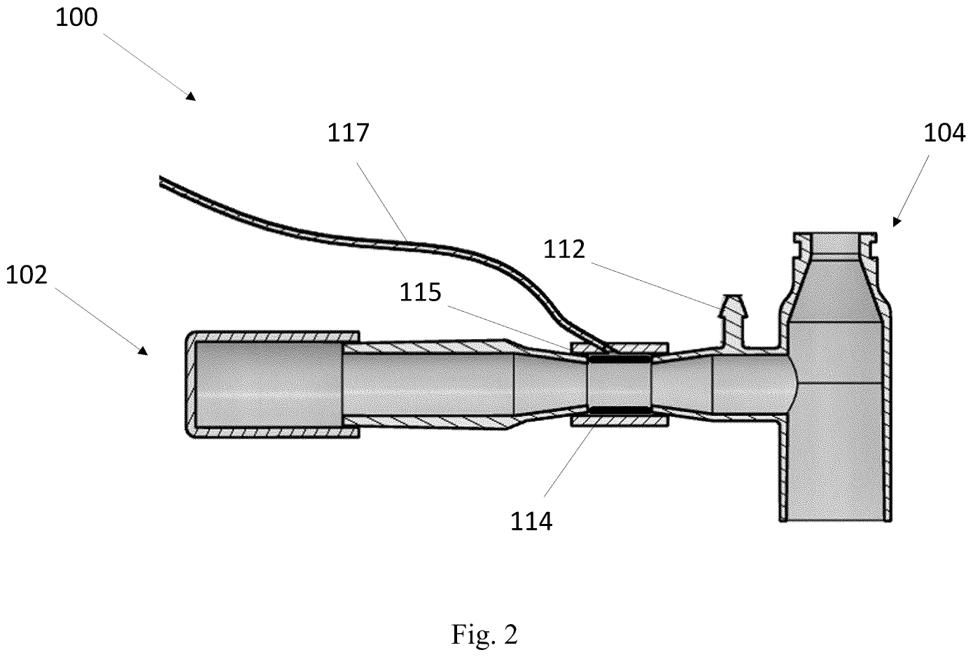

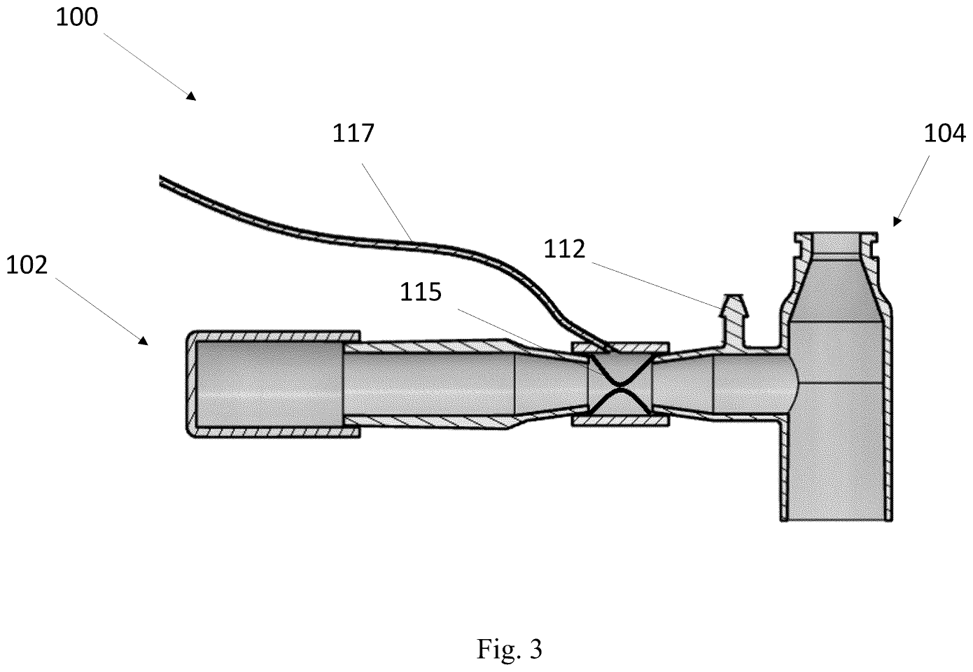

Referring now to FIG. 1, FIG. 2 and FIG. 3, an exemplary compartmentalized lung ventilation device of the present invention is shown. Device 100 comprises a proximal end 102, a distal end 104, a first lumen 106, a second lumen 108, a third lumen 110, at least one port 112, at least one flow regulator 114 and a processor.

In one embodiment, first lumen 106 is fluidly connected to a mechanical ventilator circuit 103 at proximal end 102 by any means known to one skilled in the art. In one embodiment, first lumen 106 may be connected to mechanical ventilator circuit 103 at proximal end 102 through tubing adapters per ISO 5356-1. In one embodiment, first lumen 106 may be connected to mechanical ventilator circuit 103 through any other tubing known to one skilled in the art. In one embodiment, first lumen 106 has a diameter ranging between 3-22 mm. In one embodiment, first lumen 106 may be larger than second lumen 108 and third lumen 110. In one embodiment, first lumen 106 may be smaller than second lumen 108 and third lumen 110. However, it should be appreciated that the present invention is not limited to any particular lumen diameter or lumen length, as any desired lumen length and diameter may be used as would be understood by those skilled in the art.

First lumen 106 is distally connected to second lumen 108 and a third lumen 110 and is configured to divide the flow of gas coming from mechanical ventilator 103 between second lumen 108 and third lumen 110. In one embodiment, second lumen 108 and third lumen 110 may have the same diameter. In one embodiment, second lumen 108 and third lumen 110 may have different diameters. In one embodiment, second lumen 108 may have a diameter larger than first lumen 106. In one embodiment, second lumen 108 may have a diameter smaller than first lumen 106. Second lumen 108 is configured to be fluidly connected to the left mainstem bronchus (lung 2) at distal end 104. Third lumen 110 is configured to be fluidly connected to the right mainstem bronchus (lung 1) at distal end 104. In one embodiment, second lumen 108 and third lumen 110 may be connected to lung 2 and lung 1 by any means known to one skilled in the art including but not limited to tubing.

At least one port 112 may be positioned anywhere along the length of second lumen 108 and/or third lumen 110. In one embodiment, at least one port 112 is configured to allow monitoring of regional parameters in either lung using any sensors known to one skilled in the art including but not limited to pressure sensors, EtCO₂ sensors, EtO₂ sensors, or flow sensors, and etc. In one embodiment, at least one port 112 may be positioned distal to at least one flow regulator 114.

In one embodiment, at least one port 112 is configured to allow for the use of suction or a bronchoscope while a patient is intubated. In one embodiment, at least one port 112 may comprise a barbed portion to allow easier attachment to external devices including but not limited to a bronchoscope. In one embodiment, at least one port 112 may be capped with any mechanism known to one skilled in the art to prevent leakage of gas from the patient's circuit.

At least one flow regulator 114 may be positioned anywhere along the length of second lumen 108 and/or third lumen 110 and is configured to control the flow and pressure that is provided to each lung through second lumen 108 and third lumen 110 by any mechanism known to one skilled in the art.

In one embodiment, at least one flow regulator 114 may comprise at least one inflatable member 115 positioned inside and attached to the interior surface of second lumen 108 and/or third lumen 110 (FIG. 2). In one embodiment, inflatable member 115 may be positioned within the wall of second lumen 108 and/or third lumen 110. Inflatable member 115 is fluidly connected to an external volume source of fluid through an inflation lumen 117 and is configured to be inflated/deflated. In one embodiment, the external volume source of fluid may be a manual device including but not limited to a syringe. In one embodiment, the external volume source of fluid may be a powered device including but not limited to an electric pump. In one embodiment, inflatable member 115 may comprise a valve in the flow path between the external volume source of fluid and inflatable member 115, configured to regulate the flow of medium used to inflate inflatable member 115. The valve is adapted to restrict flow and to respond to increases in pressure which may be applied by the external volume source of fluid to ensure a desired inflation rate. During deflation, the valve is configured to offer little or no resistance to medium evacuation, allowing inflatable member 115 to be deflated quickly. In one embodiment, the valve may operate with any mechanism known to one skilled in the art including but not limited to a solenoid valve, ball valve, butterfly valve, slide valve, gate valve, needle valve, pinch valve, etc. In one embodiment, inflatable member 115 may be connected to the external volume source of fluid by any means known to one skilled in the art including but not limited to a tubing. In one embodiment, inflatable member 115 may be inflated with a fluid including but not limited to saline. In one embodiment, inflatable member 115 may be inflated with a gaseous medium.

Inflatable member 115 may be inflated to a volume to completely restrict the flow and/or pressure within second lumen 108 and/or third lumen 110. In one embodiment, inflatable member 115 may be inflated to any volume so to restrict the flow and/or pressure within second lumen 108 and/or third lumen 110 between 0-100 percent by changing the effective inner diameter and surface area of each lumen.

In one embodiment, at least one flow regulator 114 may comprise a pinch mechanism including but not limited to a pinch arm, a pinch roller, etc. In one embodiment, at least one flow regulator 114 may comprise a first pinch arm and a second pinch arm. First pinch arm is positioned externally around second lumen 108. In one embodiment, first pinch arm may be positioned anywhere along the length of second lumen 108. Second pinch arm is positioned externally around third lumen 110. In one embodiment, second pinch arm may be positioned anywhere along the length of third lumen 110. The first pinch arm and the second pinch arm are configured to allow compression of second lumen 108 and third lumen 110 so that the flow/or pressure within second lumen 108 and third lumen 110 can be restricted to any percentage ranging between 0-100 percent by changing the effective diameter and surface area of each lumen.

In one embodiment, at least one flow regulator 114 may comprise at least one valve configured to provide a variable flow coefficient when positioned within the lumen. In one embodiment, the at least one valve may be positioned anywhere within the length of second lumen 108. In one embodiment, the at least one valve may be positioned anywhere within the length of third lumen 110. In one embodiment, the valve may operate with any mechanism known to one skilled in the art including but not limited to pinch valve, etc. In one embodiment, the valve may be positioned proximal to at least one port 112. In one embodiment, the valve may be able to control the flow/or pressure within second lumen 108 and third lumen 110 between 0-100 percent. In one embodiment, the valve may comprise an internal flapper wherein the angle of the flapper changes the effective flow/pressure rate of the lumen.

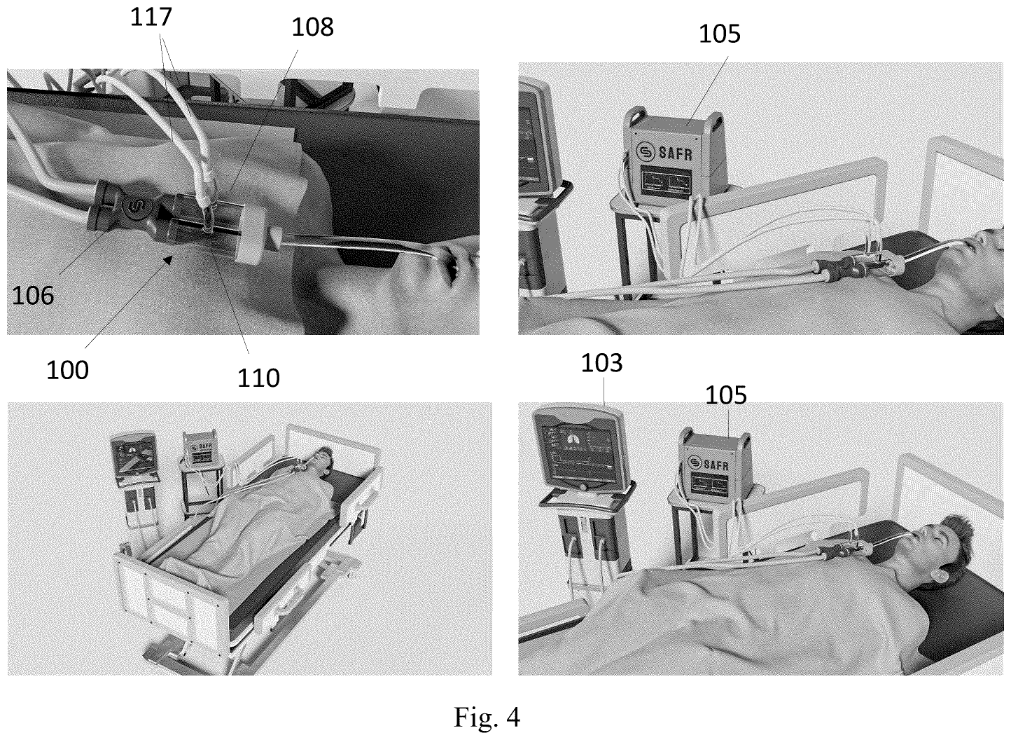

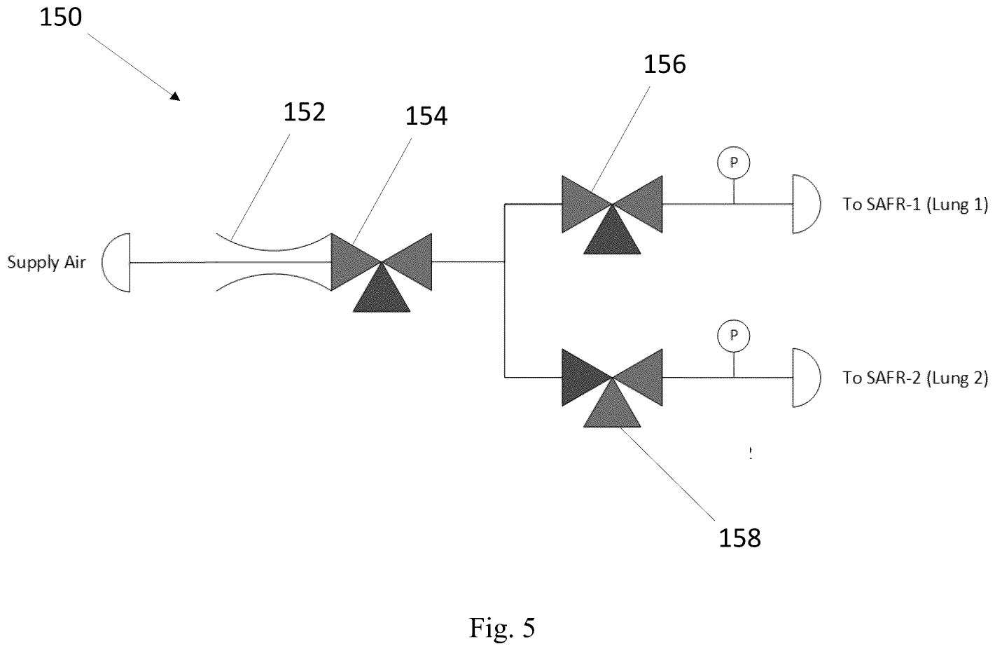

Referring now to FIG. 4, an exemplary compartmentalized lung ventilation device 100 of the present invention is shown in fluid connection to a mechanical ventilator and a subject's airway. In one embodiment, device 100 is fluidly connected to mechanical ventilator 103 at proximal end 102 and is connected to the subject's airways and lungs at distal end 104. In one embodiment, device 100 may further comprise a control module positioned within a control unit 105. Control module is configured to control the flow of air within device 100 by manipulating the flow regulators 114 contained therein, wherein the control module is represented by diagram 150 depicted in FIG. 5. As described elsewhere herein, flow regulator 114 can comprise any desired valve, including but not limited to inflatable members, solenoid valves, ball valves, butterfly valves, slide valves, gate valves, needle valves, pinch valves, and the like. For demonstration purposes, the control module is described herein in the context of inflatable members, but it should be understood that comparable control schemes are applicable to any flow regulator 114.

The control module is connected to a pressure supply of fluid media (such as a gaseous or aqueous medium) and comprises an orifice 152, a first valve control 154, a second valve control 156, a third valve control 158 and a processor. In some embodiments, the pressure supply of fluid may be an integrated air compressor or liquid pump.

Orifice 152 is positioned upstream of first valve control 154 and is configured to control the flow rate of gas entering into first valve control 154. The processor is configured to open and close the valves based on feedbacks from at least one sensor positioned in second lumen 108 and third lumen 110, such as the pressure sensors shown in control module diagram 150 indicated by the (P) downstream from the valve controls. First valve control 154 is positioned upstream of second valve control 156 and third valve control 158.

Second valve control 156 is configured to control at least one flow regulator 114 of device 100. In one exemplary embodiment, wherein the at least one flow regulator 114 comprises an inflatable member 115 as described elsewhere herein, second valve control 156 modulates fluid flow from the pressure supply of fluid through inflation lumen 117 into inflatable member 115. This alters the effective cross-sectional area of the second lumen 108 and changes the volumetric flow rate of gas into lung 1 from mechanical ventilator 103.

Third valve control 158 is configured to control at least one flow regulator 114 of device 100. In one exemplary embodiment, wherein the at least one flow regulator 114 comprises an inflatable member 115 as described elsewhere herein, third valve control 158 modulates fluid flow from the pressure supply of fluid through inflation lumen 117 into inflatable member 115. This alters the effective cross-sectional area of the third lumen 110 and changes the volumetric flow rate of gas into lung 2 from mechanical ventilator 103.

Referring now to FIG. 5, control module 150 is shown in a configuration wherein first valve control 154 and second valve control 156 are open, thereby directing supply fluid to inflatable member 115 of second lumen 108 via an inflation lumen 117. Accordingly, flow within second lumen 108 is restricted, which increases restriction of air from mechanical ventilator 103 to lung 1. Third valve control 158 is closed, thereby restricting supply fluid from flowing into inflatable member 115 of third lumen 110 and providing no restriction in gas flow to lung 2 through third lumen 110.

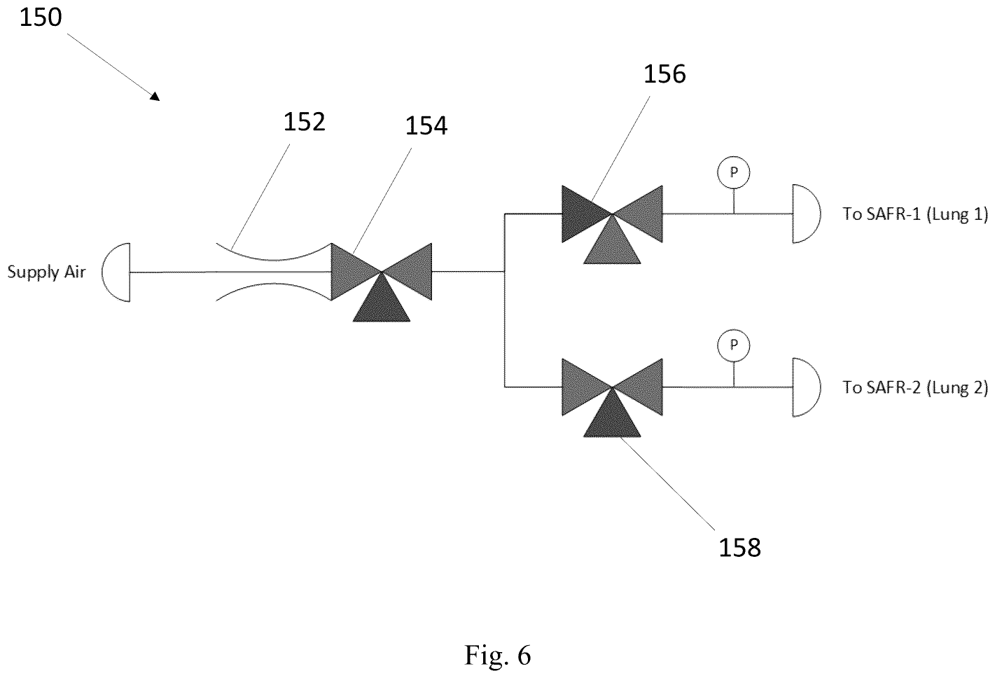

Referring now to FIG. 6, control module 150 is shown in a configuration wherein first valve control 154 and third valve control 158 are open, thereby directing supply fluid to inflatable member 115 of third lumen 110 via an inflation lumen 117. Accordingly, flow within third lumen 110 is restricted, which increases restriction of air from mechanical ventilator 103 to lung 2. Second valve control 156 is closed, thereby restricting supply fluid from flowing into inflatable member 115 of second lumen 108 and providing no restriction in gas flow to lung 1 through second lumen 108.

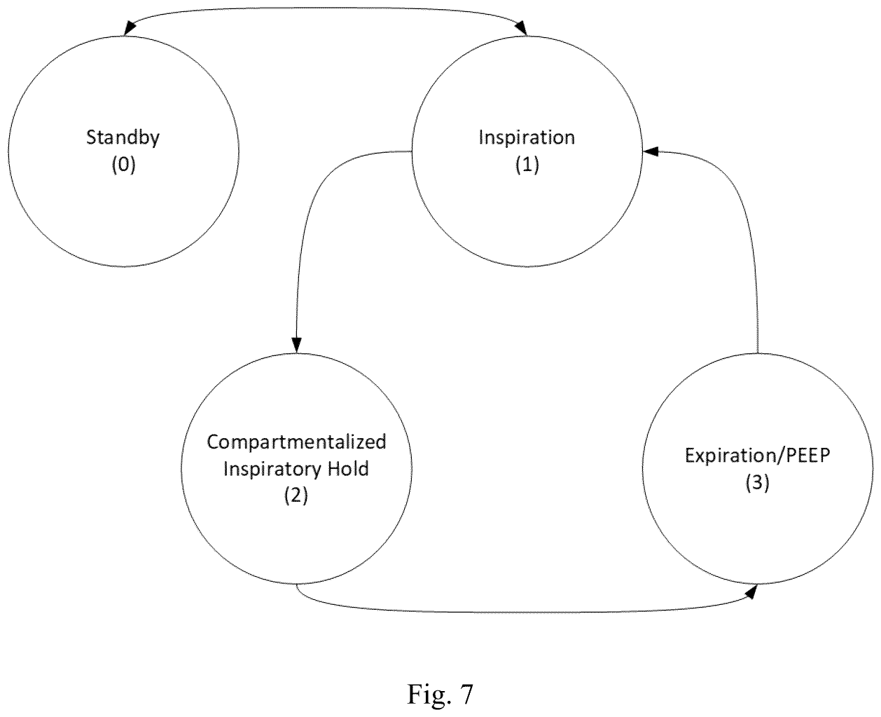

In one embodiment, these configurations (FIG. 5 and FIG. 6) may be used during an inspiration phase (FIG. 7). In an inspiration phase, gas is actively flowing from the mechanical ventilator 103 into the lungs of the patient. During this phase, the processor is configured to alter the distribution of volume between the two lungs in a non-pathophysiologic way. This is done by regulating the flow of gas through either second lumen 108 or third lumen 110 into the patient's lungs.

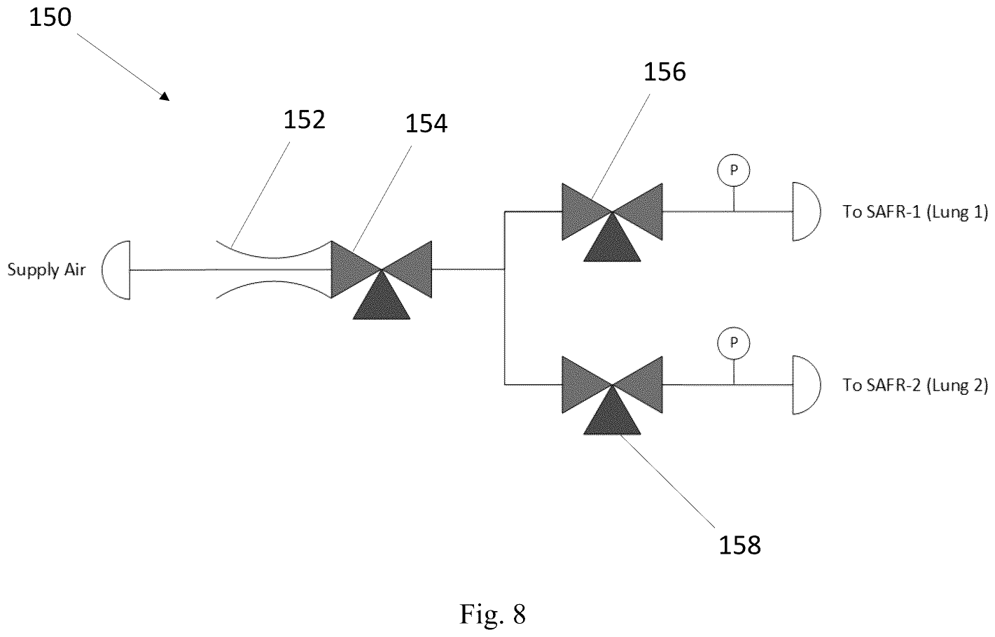

Referring now to FIG. 8, control module 150 is shown in a configuration wherein first valve control 154, second valve control 156, and third valve control 158 are all open. Supply fluid is thereby directed to inflatable members 115 of both second lumen 108 and third lumen 110 via respective inflation lumens 117. Accordingly, flow within each of second lumen 108 and third lumen 110 is restricted, which increases restriction of air from mechanical ventilator 103 to lung 1 and lung 2. This configuration may be used during the compartmentalized inspiratory hold phase or after appropriate PEEP is achieved during expiration (FIG. 7). Compartmentalized inspiratory hold phase occurs for a short duration after the end of inspiration and may only be 250 milliseconds long. In this state, a no-flow condition is created between the different regions of the lungs or between lung 1 and lung 2. The compartmentalized inspiratory hold phase creates isolated pressure volumes where the plateau pressure and EtCO₂ levels can be independently measured in either lung. In expiration, the gas is actively flowing from the patient's lungs through the expiratory module of mechanical ventilator 103. During this phase, the control module is configured to work to ensure there is no auto-PEEP or excess buildup of pressure inside of the regions of the patient's lungs. In another embodiment, device 100 may work to achieve an independent PEEP setpoint in either lung, based on clinical needs of the patient (FIG. 7).

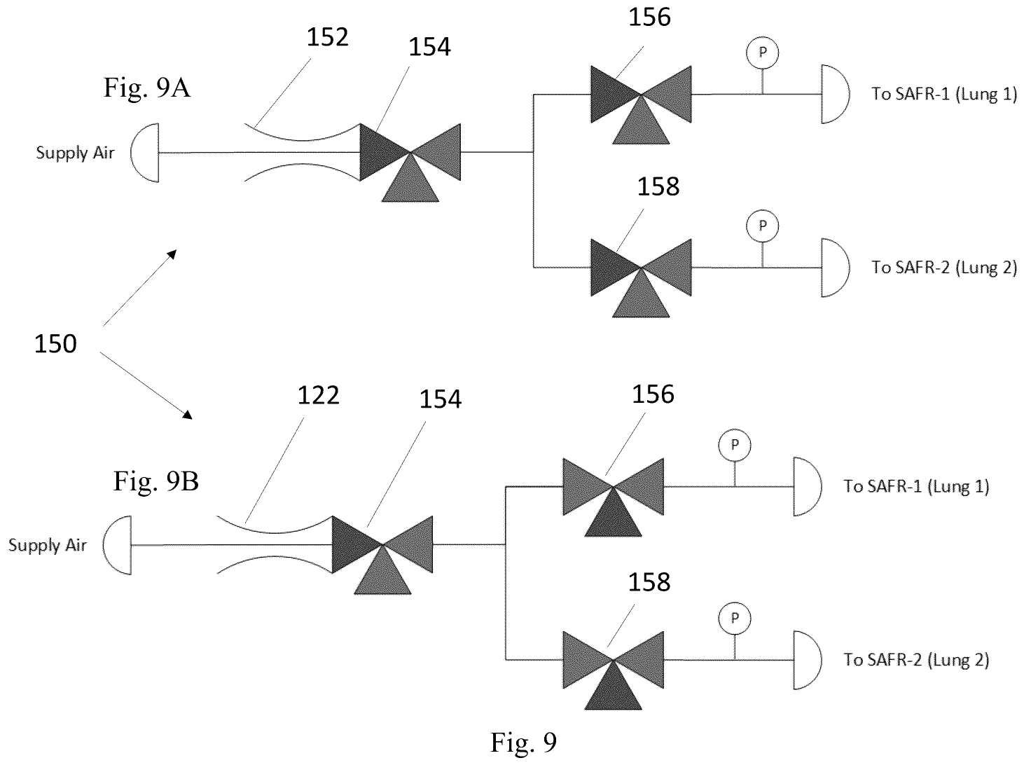

Referring now to FIG. 9A and FIG. 9B, control module 150 is shown in configurations wherein first valve control 154 is closed. Supply fluid is vented out of inflatable members 115 of both second lumen 108 and third lumen 110, thereby deflating the inflatable members 115. In the top diagram, supply fluid is vented directly out of the closed second valve control 156 and third valve control 158, while in the bottom diagram, supply fluid backs out inflation lumens 117 to be vented out of the closed first valve control 154. In these configurations, flow restrictions in second lumen 108 and third lumen 110 are removed due to the deflation of inflatable members 115, thereby permitting flow of gas from mechanical ventilator 103 to both lung 1 and lung 2. These configurations may be used during the expiration phase (FIG. 7).

In some embodiments, an open valve control energizes a respective flow regulator, and a closed valve control deenergizes a respective flow regulator. In some embodiments, an open valve control deenergizes a respective flow regulator, and a closed valve control energizes a respective flow regulator.

Device 100 may further comprise a standby phase, where the processor is waiting for active commands to adjust the flow of gas to various regions of the lungs (FIG. 7).

Attached sensors and at least one flow regulator 114 may be communicatively connected to a processor. The processor is configured to perform computing steps, including sensor reading steps for example obtaining one or more samples from at least one port 112, and/or parameter adjustment steps, comprising sending data to any sensors or at least one flow regulator 114 to activate/deactivate the sensors or adjust flow in each lumen. In some embodiments, the processor may include actuation steps performed in response to particular values of at least one sensor measurement, for example activating at least one flow regulator 114 to adjust flow/and or pressure in first lumen 106, second lumen 108 and/or third lumen 110.

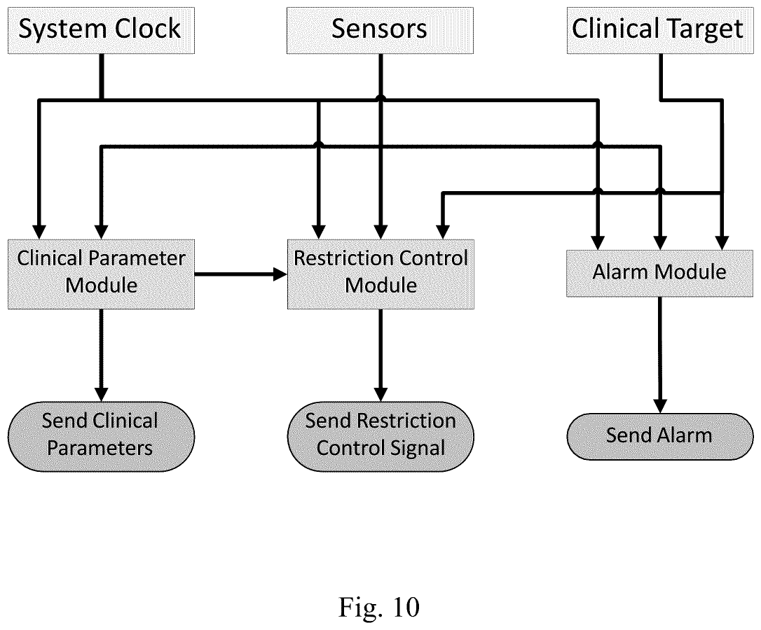

In one embodiment, the processor comprises an embedded software. In one embodiment, the software comprises a restriction control module (RCM) configured to use input values from at least one sensor as a way to determine what regulations are needed in either first lumen 106, second lumen 108 and/or third lumen 110 to achieve the clinical target (FIG. 10). In one embodiment, the software comprises a clinical parameter module (CPM) configured to measure clinical parameters of a patient using data from at least one sensor and data from the clock as a way to determine clinical parameters of the patient, including, but not limited to, respiratory rate, inspiratory pressures, plateau pressures, delivered tidal volume, lung compliance, and more (FIG. 10). In one embodiment, the software comprises an alarm module (AM) configured to ensure device 100 is safe and is able to protect the patient (FIG. 10). In one embodiment, software comprises a threshold value for flow and clinical parameters. In one embodiment, the alarm module is configured to compare the data from the patient with the standard values set by the clinicians and alarm the user if clinical parameters fall below or above the threshold. In one embodiment, the alarm module may use any alarm or alert known to one skilled in the art including but not limited to visual, auditory, sensory or any combination thereof.

In one embodiment, the processor is configured to use the data from at least one sensor to determine the desired flow regulation inside each lumen. In one embodiment, a closed loop algorithm may be used to implement a proportional-integral-derivative controller (PID controller) for achieving the correct flow rate of gas through second lumen 108 and/or third lumen 110 based on the distal parameters of the lungs. In one embodiment, the processor enables direct intrathoracic observing and quantifying functions of left and right lungs simultaneously and independently. In one embodiment, the processor enables regulating gas flow to either the left or the right lung based on physiologic or pathologic differences between the lungs. In one embodiment, the processor may use data collected from at least one sensor including but not limited to pressure sensors, gas concentration sensors, such as EtCO₂ sensors, imaging scans of the lungs, or other data, to observe and quantify functions of left and right lungs. This data is parameterized in order to control at least one flow regulator 114 and thus provide gas to the left and right lungs of the patient in an optimized and tailored manner based on pathophysiological needs of each lung to reduce lung injury and improve patient care.

In one embodiment, the processor may include advanced algorithms configured to determine the impact of factors such as delivered volume, respiratory rate, gas transfer rates, airway pressures achieved during inspiration and expiration, and other factors which can provide real-time clinical feedback to clinicians to allow them to provide the best care to a patient. In one embodiment, the advanced algorithm may be a machine learning framework configured to provide predictive analytics to a clinician and improve patient care. In one embodiment, advanced software algorithms may be configured to train the device in supervised machine learning, where the data from the device is compared to traditional lung performance test results and other clinical tests. In one embodiment, advanced software algorithms may be configured to train the device in unsupervised machine learning, where time-based data from the device is used to develop a model based on how future lung performance is impacted by past lung performance. In one embodiment, the processor may be configured to provide predictive care outcomes and personalized medicine for individual patients based on data acquired from at least one sensor.

In one embodiment, the processor may comprise a user interface having keypads and a display configured to receive user inputs, such as through manipulation of keys of the keypad and provide output to a user by textual, numeric, and graphical presentation on the display and/or by aural output through audio. In one embodiment, the processor may receive input by any other means known to one skilled in the art, such as by audio inputs.

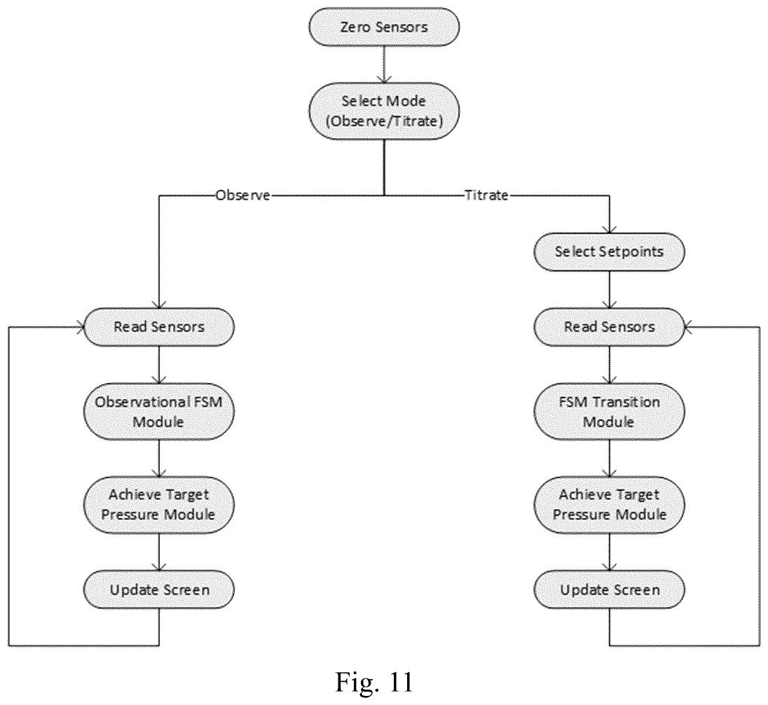

Referring now to FIG. 11 through FIG. 17, functional block diagrams of various hardware and software modules are shown. In one embodiment, the software module may start by zeroing all of the sensors (FIG. 11). In one embodiment, a user may be prompted to select a mode of operation including but not limited to observe mode, titrate mode, etc. (FIG. 11). In observation mode, no changes are made to the flow of gas to regions of the lungs. In titrate mode, the processor may use a closed-loop control module to actively adjust the flow of gas to regions of the lungs. In one embodiment, various software modules are executed based on the desired mode of operation of the clinician and user. In one embodiment, as shown in FIG. 11, observe mode may include performing a set of steps in a finite or infinite loop, for example the steps of reading one or more sensors, executing or calling an observational FSM module, executing or calling an achieve target pressure module, and updating the screen. In one embodiment, a titrate mode may include the steps of selecting one or more setpoints, either via a configuration or user input, reading one or more sensors, executing or calling an FSM transition module, executing or calling an achieve target pressure module, updating the screen, and then repeating the steps beginning from the step of reading one or more sensors.

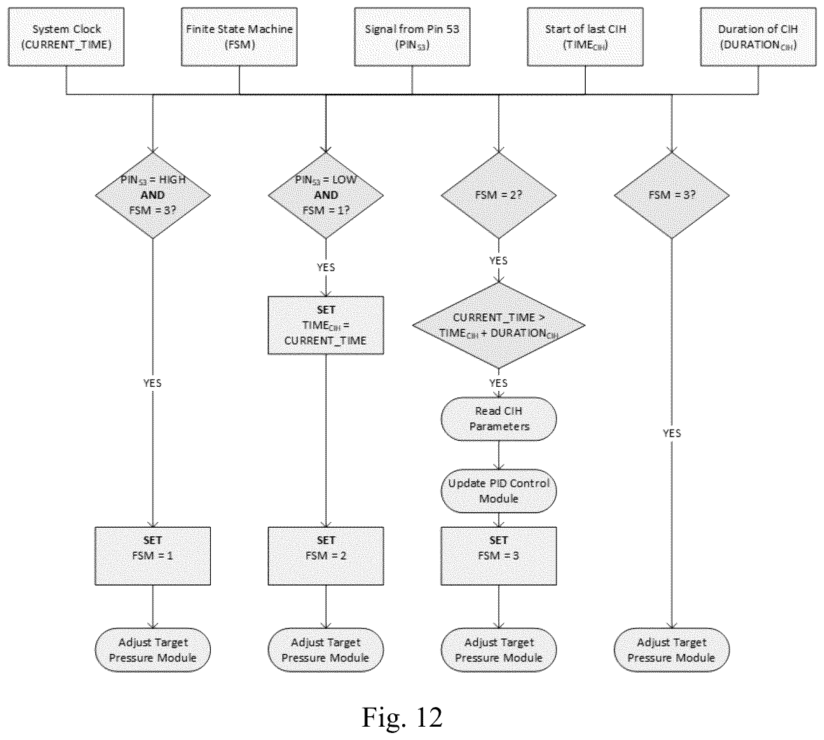

In one embodiment, the software module may comprise a Finite State Machine (FSM) Transition Module (see FIG. 12), which may be a part of the titrate control block (see FIG. 11). An exemplary FSM transition module comprises multiple inputs including but not limited to the system clock, the state of the finite state machine, a signal pin from the mechanical ventilator, and parameters related to a compartmentalized inspiratory hold (CIH), for example the start time and/or duration of a CIH. In one embodiment, the signal pin is configured to provide insight into whether the ventilator is in inspiration phase. Based on these inputs, the module is configured to move between states of the internal finite state machine according to the state diagram shown in FIG. 12, and perform the functions of the various states including but not limited to adjusting the target pressure, as depicted in FIG. 12, where appropriate.

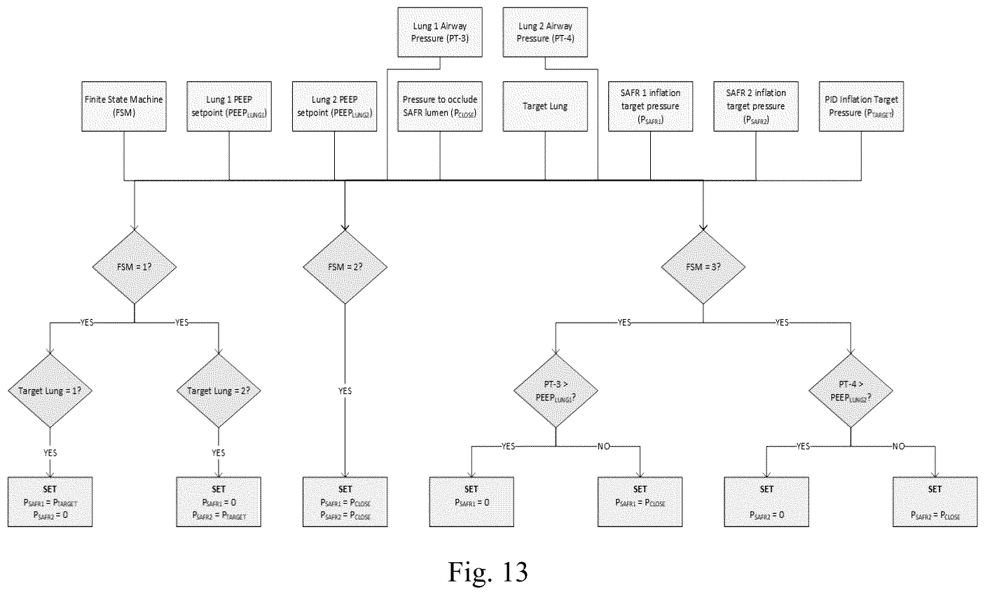

In one embodiment, the software module may comprise an Achieve Target Pressure Module (FIG. 13) for example as part of the FSM depicted in FIG. 12. In one embodiment, the module has multiple inputs, including but not limited to a state of the finite state machine, the setpoints of the target PEEP value for Lung 1 and/or Lung 2 (for example the volume and pressure required inside of inflatable member 115 to completely occlude the flow path to either second lumen 108 and/or third lumen 110), the target lung to adjust the flow to, the target inflation volume and pressure of inflatable member 115, the inflation target volume and pressure as calculated by a closed-loop PID controller, and the airway pressure in Lung 1 and Lung 2. The exemplary FSM shown in FIG. 13 moves between states based on the state diagram shown. The state machine is able to adjust the target volume and pressure for inflatable member 115 to Lung 1 and Lung 2. In one exemplary embodiment, if the state of the device is 1 (inspiration) and the target lung is Lung 1, the processor may set the target inflation volume and pressure equal to the target volume and pressure determined by the closed-loop PID controller. In another exemplary embodiment, if the state of device 100 is compartmentalized inspiratory hold, then the processor may set the target inflation volume and pressure of both inflatable member 115 positioned in second lumen 108 and third lumen 110 equal to the volume and pressure required to fully occlude the flow paths to both lungs, thus providing a no-flow condition in the lungs.

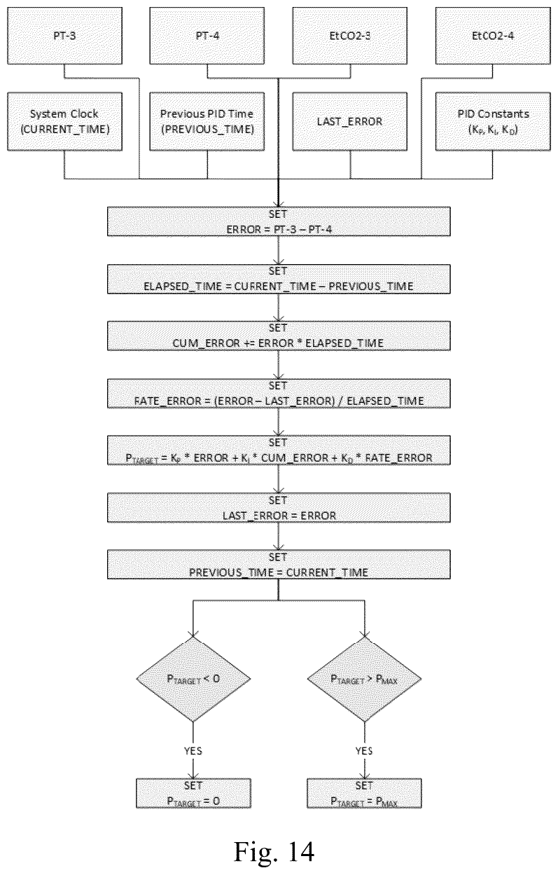

In one embodiment, the software module may comprise a Proportional-Integral-Derivative (PID) Controller Module like the one shown in FIG. 14. In one embodiment, this module comprises multiple inputs, including but not limited to the sensors that may be used for determining the error of the system, such as airway pressure and airway EtCO₂. In one embodiment, at least one input may be required for the PID algorithm. Some or all of these inputs may be used to calculate the error. In one embodiment, the error may be the difference in airway pressure between Lung 1 and Lung 2 (PT-3 minus PT-4). In other embodiments, the clinician may desire for the pressure or EtCO₂ between the lungs to not be equal but also not be the same as pathophysiologic baseline. In this embodiment, the module calculates a new volume or pressure target based on the error, the cumulative error, and the error rate. PID coefficients are also used in this calculation. If the calculated target volume or pressure is below zero, it is set to zero. If the calculated target volume or pressure is above the maximum allowable volume and pressure, it is set to the maximum allowable volume and pressure.

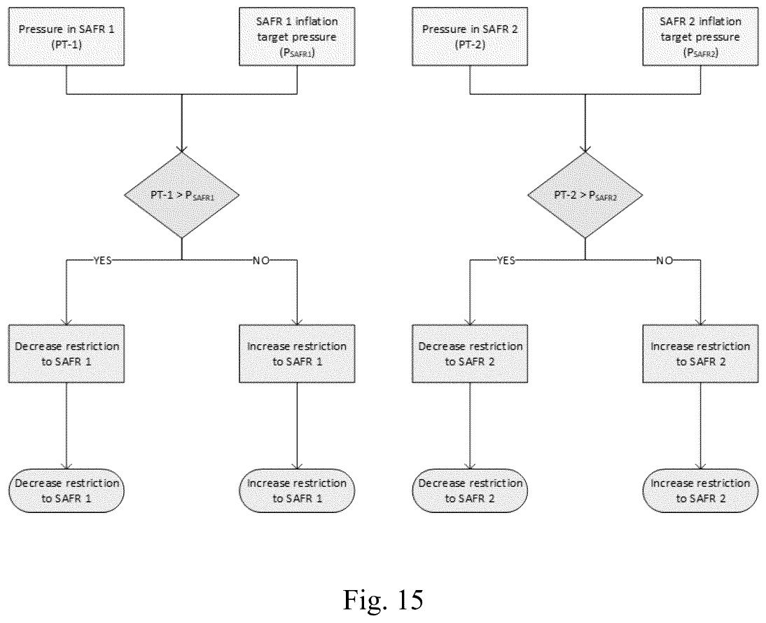

In one embodiment, the software module may comprise an Achieve Target Pressure Module (for example as shown in FIG. 15). In one embodiment, this module may comprise multiple inputs, including but not limited to the current volume and pressure in inflatable member 115, restricting the flow of gas to Lung 1 and Lung 2 and the target volume and pressure in inflatable member 115 restricting the flow of gas to Lung 1 and Lung 2. In one embodiment, the module is configured to perform calculations to determine whether the current volume and pressure in inflatable member 115 is higher or lower than the target volume and pressure and based on the outcome of those calculations, the processor may either decrease or increase the volume and pressure in inflatable member 115 to achieve the target volume and pressure. In one embodiment, the Achieve Target Pressure Modules for Lung 1 and Lung 2 may be identical, i.e., identical inputs to the Lung 1 and Lung 2 modules will yield identical behavior. In one embodiment, one or more parameters of an Achieve Target Pressure Module for Lung 1 may differ from one or more parameters of an Achieve Target Pressure Module for Lung 2, for example based on unique physiological constraints of the patient.

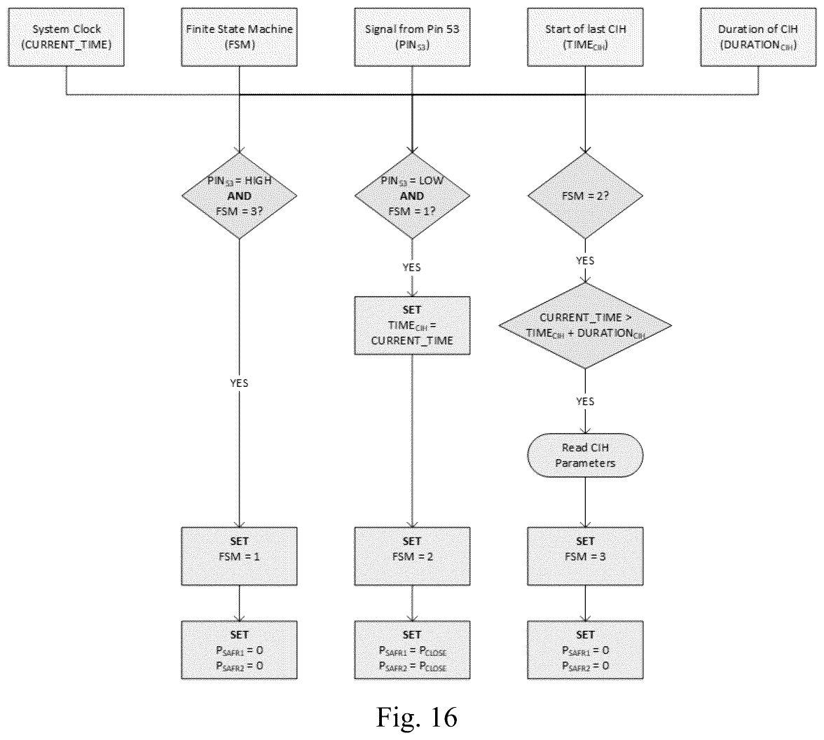

In one embodiment, the software module may comprise an Observational Finite State Machine (FSM) Module like the one shown in FIG. 16, which may be integrated into an observation control block for example as shown in FIG. 11. In one embodiment this module may have multiple inputs, including but not limited to a system clock, a state of the finite state machine, a signal pin from the mechanical ventilator configured to provide insight into whether the ventilator is in inspiration phase, the start time of the last compartmentalized inspiratory hold phase, and the duration of a compartmentalized inspiratory hold phase. Based on these inputs, the module may adjust the state of the internal finite state machine if appropriate. Before or after adjusting the state of the finite state machine, the system may adjust one or more parameters of the device as appropriate, for example the target volume and pressure in the inflatable member 115 as appropriate for observation of patient regional pathophysiology.

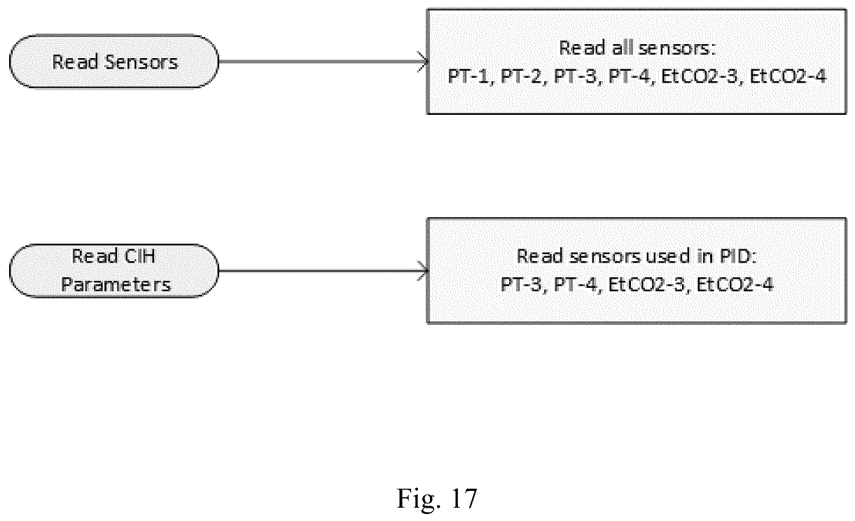

In one embodiment, the software module may comprise a sensor reading module (FIG. 17). In one embodiment, this module may have at least two sub-functions. In one embodiment, a first sub-function may be configured to read a first subset of the sensors, for example all sensors as depicted in the top diagram of FIG. 17. In one embodiment, a second sub-function may be configured to read a second subset of the sensors, for example only the sensors whose values are used as inputs to the PID controller (see e.g., FIG. 14).

In one embodiment, device 100 may be positioned anywhere between mechanical ventilator 103 and the patient's lungs. In one embodiment, device 100 may be positioned distal to mechanical ventilator 103. In one embodiment, device 100 may be positioned within the patient's body.

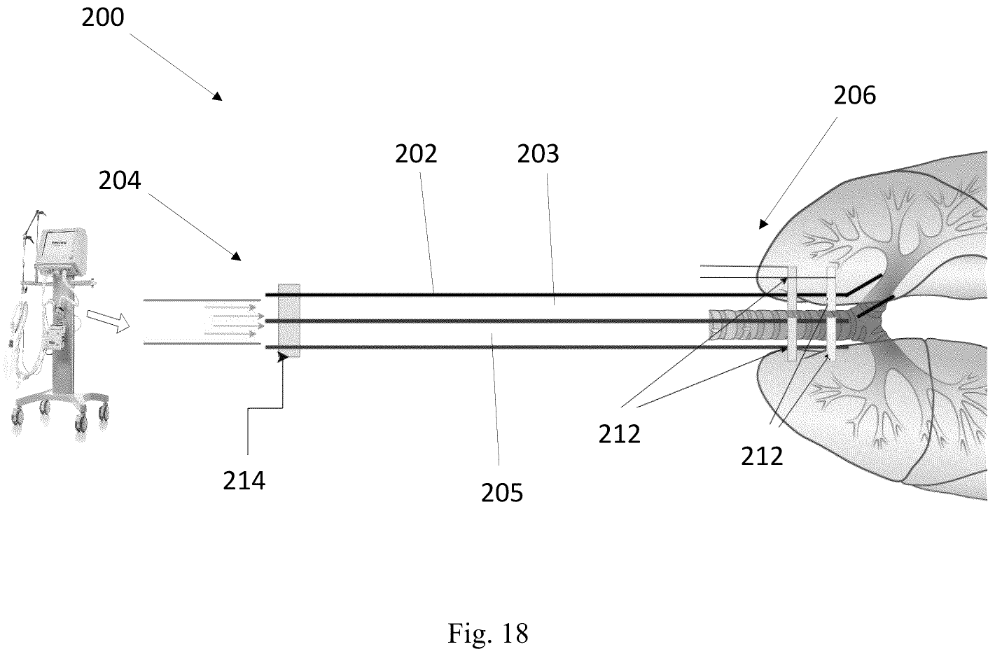

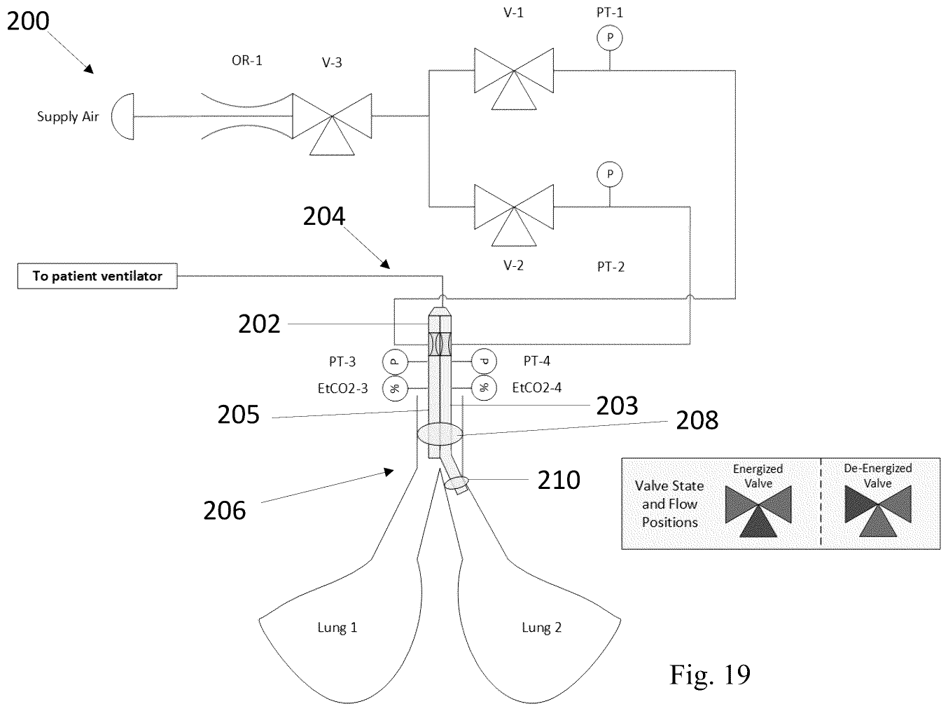

Referring now to FIG. 18 and FIG. 19, an exemplary compartmentalized lung ventilation device 200 of the present invention is shown. Device 200 comprises a double lumen endotracheal tube 202 having a proximal end 204, a distal end 206, a first balloon 208, a second balloon 210, at least one sensor 212, at least one flow regulator 214 and a processor.

Double lumen endotracheal tube 202 comprises a first lumen 203 and a second lumen 205 extending from proximal end 204 toward distal end 206. In one embodiment, first lumen 203 and second lumen 205 may have the same diameter. In one embodiment, first lumen 203 has a larger diameter than second lumen 205. In one embodiment, first lumen 203 has a smaller diameter than second lumen 205. In one embodiment, first lumen 203 and second lumen 205 have a diameter ranging approximately between 4-7 mm. In one embodiment, first lumen 203 and second lumen 205 may have a length ranging approximately between 30-60 cm. However, it should be appreciated that the present invention is not limited to any particular lumen diameter or lumen length, as any desired lumen length and diameter may be used as would be understood by those skilled in the art. In one embodiment, first lumen 203 has a longer length than second lumen 205. In one embodiment, first lumen 203 has a smaller length than second lumen 205.

Double lumen endotracheal tube 202 may be made from any material known to one skilled in the art including but not limited to plastic. In one embodiment, double lumen endotracheal tube 202 may be flexible.

First lumen 203 and second lumen 205 are each fluidly connected to a mechanical ventilator circuit at proximal end 204 by any means known to one skilled in the art. In one embodiment, first lumen 203 and second lumen 205 may be connected to a mechanical ventilator circuit at proximal end 204 through free standing tubes. In one embodiment, first lumen 203 and second lumen 205 may be connected to a mechanical ventilator circuit at proximal end 204 through tubing adapters per ISO 5356-1. In one embodiment, first lumen 203 and second lumen 205 may be connected to a mechanical ventilator circuit through any other tubing known to one skilled in the art. In one embodiment, the connecting tubing may have a smaller diameter than first lumen 203 and second lumen 205. In one embodiment, the connecting tubing may have a larger diameter than first lumen 203 and second lumen 205.

First lumen 203 is fluidly connected to the left mainstem bronchus (lung 2) at distal end 206. In one embodiment, first lumen 203 may be curved at a distal end to allow intubation of the left mainstem bronchus. Second lumen 205 is fluidly connected to the right mainstem bronchus (lung 1) at distal end 206. In one embodiment, second lumen 205 may be curved at a distal end to allow intubation of the right mainstem bronchus. However, it should be appreciated that the present invention is not limited to any particular lumen angle, as any desired lumen angle may be used as would be understood by those skilled in the art.

First balloon 208 is positioned externally around double lumen endotracheal tube 202 at distal end 206 and is configured to mechanically secure device 200 inside the patient's airway. First balloon 208 is fluidly connected to an external volume source of fluid to allow inflating and deflating the balloon. In one embodiment, the external volume source of fluid may be a manual device including but not limited to a syringe. In one embodiment, the external volume source of fluid may be a powered device including but not limited to an electric pump. First balloon 208 may be connected to the external pressure source by any means known to one skilled in the art. In one embodiment, first balloon 208 may be inflated with a fluid including but not limited to saline. In one embodiment, first balloon 208 may be inflated with a gaseous medium.

Second balloon 210 is positioned externally around first lumen 203 at distal end 206, distal to the curvature and is configured to mechanically secure device 200 inside the patient's airway. Second balloon 210 is fluidly connected to an external pressure source to allow inflating and deflating the balloon. In one embodiment, the external pressure source may be a manual device including but not limited to a syringe. In one embodiment, the external pressure source may be a powered device including but not limited to an electric pump. In one embodiment, second balloon 210 may comprise a valve in the flow path between the external pressure source and second balloon 210, configured to regulate the flow of medium used to inflate second balloon 210. The valve is adapted to restrict flow and to respond to increases in pressure which may be applied by the external pressure source to ensure a desired inflation rate. During deflation, the valve is configured to offer little or no resistance to medium evacuation, allowing the balloon to be deflated quickly. In one embodiment, the valve may operate with any mechanism known to one skilled in the art including but not limited to a sliding action valve, gate valve, etc. In one embodiment, second balloon 210 may be connected to the external pressure source by any means known to one skilled in the art. In one embodiment, second balloon 210 may be inflated with a fluid including but not limited to saline. In one embodiment, second balloon 210 may be inflated with a gaseous medium. In one embodiment, second balloon 210 may be smaller than first balloon 208.

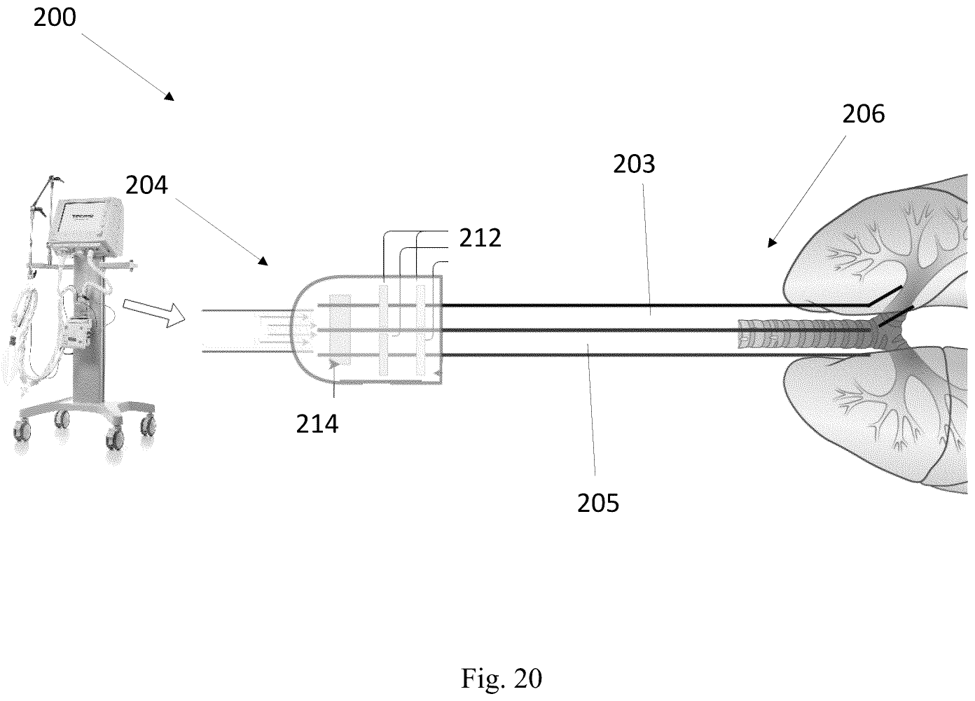

At least one sensor 212 may be positioned anywhere throughout the length of double lumen endotracheal tube 202. In one embodiment, at least one sensor 212 may be positioned anywhere throughout the length of first lumen 203 and/or second lumen 205. In one embodiment, at least one sensor 212 may be positioned at distal end 206 of first lumen 203 and/or second lumen 205 (FIG. 18). In one embodiment, at least one sensor 212 may be positioned at proximal end 204 of first lumen 203 and/or second lumen 206 (FIG. 20). In one embodiment, at least one sensor 212 may be positioned within the wall of first lumen 203 and/or second lumen 205. In one embodiment, at least one sensor 212 may be positioned within the patient trachea or bronchus, or directly downstream of at least one flow regulator 214 outside of the patient body. In one embodiment, at least one sensor 212 may be positioned proximal to first balloon 208. In one embodiment, at least one sensor 212 may be positioned proximal to second balloon 210.

In one embodiment, at least one sensor 212 includes but is not limited to: pressure sensors, EtCO₂ sensors, EtO₂ sensors, flow sensors, and etc. In one embodiment, at least one sensor 212 may be configured to collect physiologic or pathologic data, such as plateau pressures, lung pressures, EtCO₂ levels configured to measure hemodynamic response of the lungs and changes in alveolar recruitment, or other values from either within the respiratory tract of a patient, such as the bronchus or trachea or from within the patient's body.

At least one flow regulator 214 is configured to control the flow and pressure that is provided to each lung through first lumen 203 and second lumen 205 by any mechanism known to one skilled in the art. In one embodiment, the at least one flow regulator 214 may be able to control the flow/or pressure within first lumen 203 and second lumen 205 between 0-100 percent. In one embodiment, the at least one flow regulator 214 may be positioned anywhere within the length of first lumen 203. In one embodiment, the at least one flow regulator 214 may be positioned anywhere within the length of second lumen 205. In one embodiment, the at least one flow regulator 214 may be positioned proximal to at least one sensor 212.

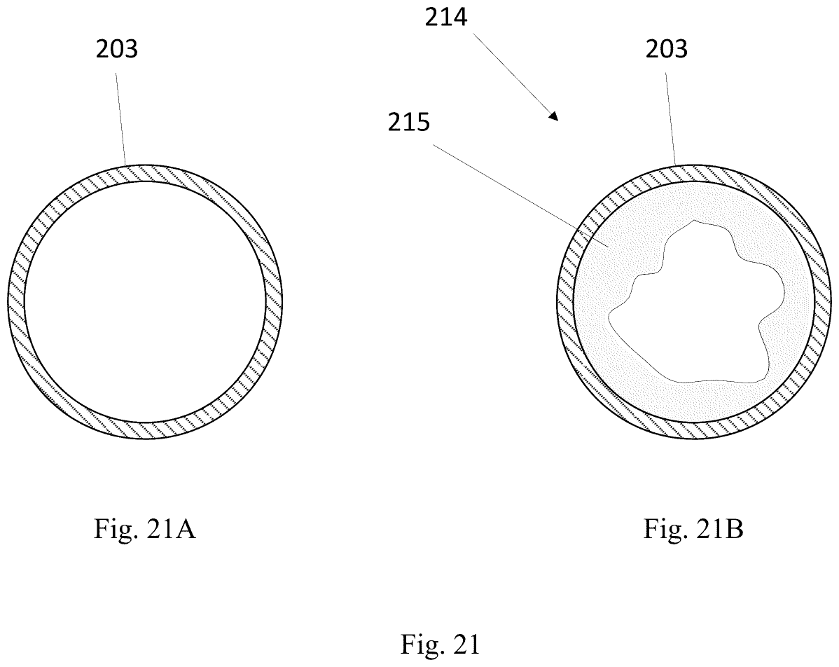

In one embodiment, at least one flow regulator 214 may comprise at least one inflatable member 215 positioned inside attached to the interior surface of first lumen 203 and/or second lumen 205 (such as the embodiment depicted in FIG. 21A and FIG. 21B). In one embodiment, inflatable member 215 may be positioned within the wall of first lumen 203 and/or second lumen 205. Inflatable member 215 is fluidly connected to an external pressure source and is configured to be inflated/deflated. In one embodiment, the external pressure source may be a manual device including but not limited to a syringe. In one embodiment, the external pressure source may be a powered device including but not limited to an electric pump. In one embodiment, inflatable member 215 may comprise a valve in the flow path between the external pressure source and inflatable member 215, configured to regulate the flow of medium used to inflate inflatable member 215. The valve is adapted to restrict flow and to respond to increases in pressure which may be applied by the external pressure source to ensure a desired inflation rate. During deflation, the valve is configured to offer little or no resistance to medium evacuation, allowing inflatable member 215 to be deflated quickly. In one embodiment, the valve may operate with any mechanism known to one skilled in the art including but not limited to a sliding action valve, gate valve, etc. In one embodiment, inflatable member 215 may be connected to the external pressure source by any means known to one skilled in the art including but not limited to a tubing. In one embodiment, inflatable member 215 may be inflated with a fluid including but not limited to saline. In one embodiment, inflatable member 215 may be inflated with a gaseous medium. Inflatable member 215 may be inflated to a volume to restrict flow completely or partially and/or pressure within first lumen 203 and/or second lumen 205 by changing the effective inner diameter and surface area of each lumen.

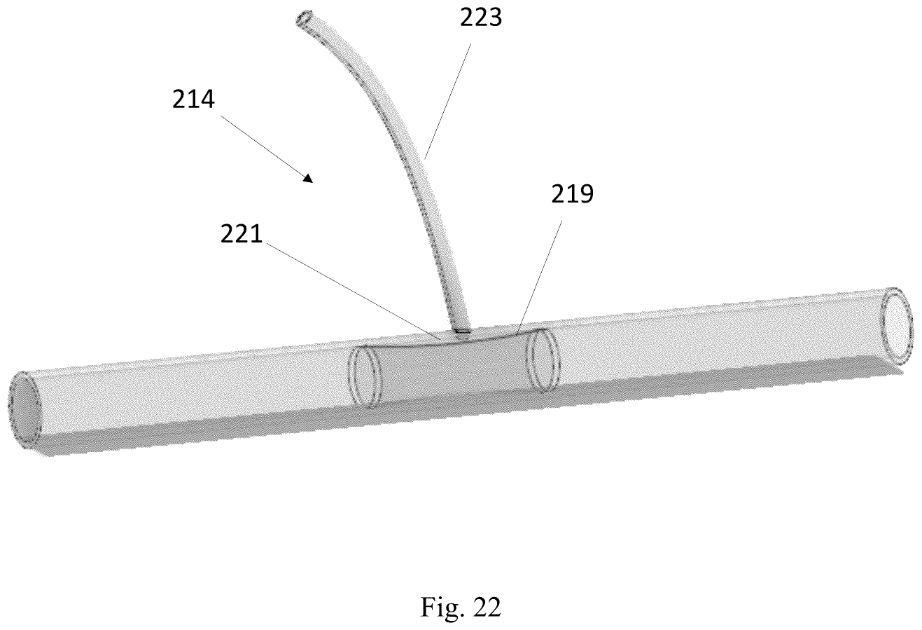

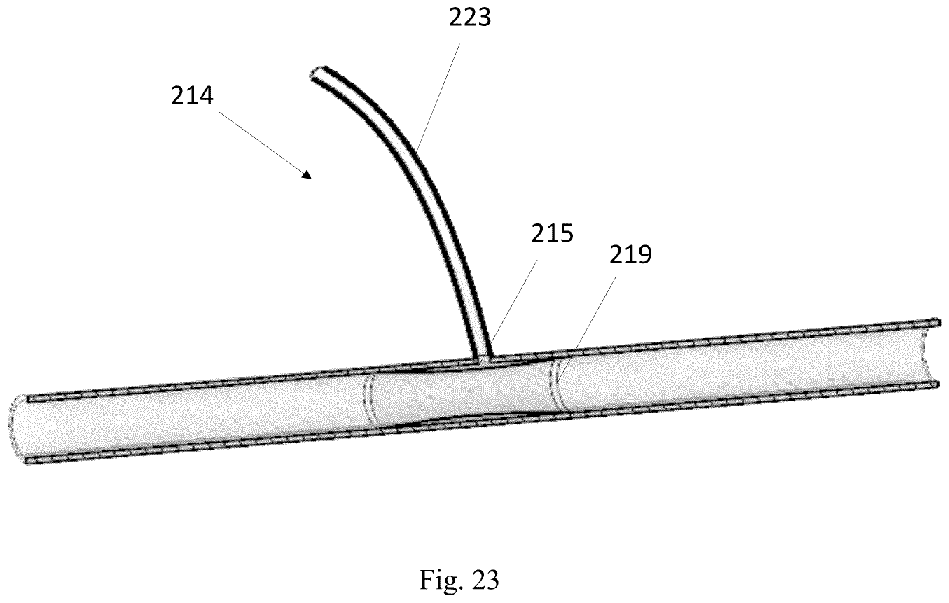

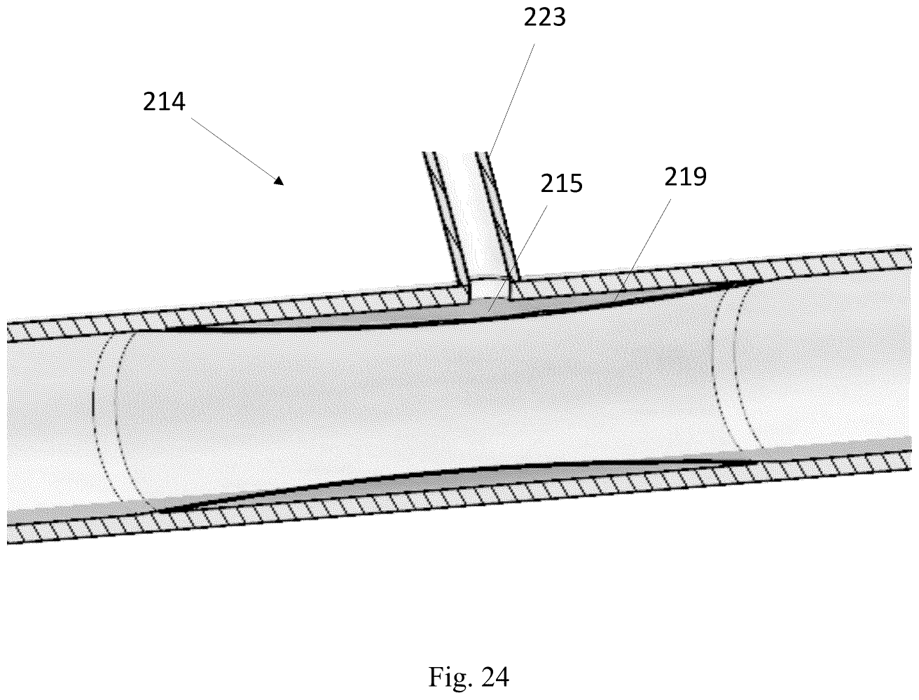

Referring now to FIG. 22 through FIG. 24, another exemplary flow regulator 214 of the present invention is shown. In one embodiment, at least one flow regulator 214 may comprise an inner lumen 219 and a thin film 221. Inner lumen 219 is positioned within first lumen 203 and/or second lumen 205. In one embodiment, inner lumen 219 may have any diameter ranging between approximately 4-7 mm. In one embodiment, inner lumen 219 has a smaller diameter than first lumen 203 and/or second lumen 205. In one embodiment, inner lumen 219 may have a length ranging between approximately 30-60 mm. However, it should be appreciated that the present invention is not limited to any particular lumen diameter or lumen length, as any desired lumen length and diameter may be used as would be understood by those skilled in the art.

Thin film 221 is bonded to the inner diameter of first lumen 203 and/or second lumen 205 and is positioned between inner lumen 219 and first lumen 203 and/or second lumen 205. Thin film 221 is fluidly connected to an external pressure source through an inflation lumen 223 that is configured to inflate and deflate the thin film with a medium. In one embodiment, the medium may be any fluid including but not limited to saline. In one embodiment, the medium may be any gas including but not limited to air. Thin film 219 may be inflated to a volume to restrict the flow and/or pressure completely or partially within first lumen 203 and/or second lumen 205 by changing the effective diameter and surface area of each lumen.

In one embodiment, at least one flow regulator 214 may comprise a pinch mechanism including but not limited to a pinch arm, a pinch roller, etc. In one embodiment, at least one flow regulator 214 may comprise a first pinch arm and a second pinch arm. First pinch arm is positioned externally around first lumen 203. Second pinch arm is positioned externally around second lumen 205. The first pinch arm and the second pinch arm are configured to allow compression of first lumen 203 and second lumen 205 so that the flow/or pressure within first lumen 203 and second lumen 205 can be partially or completely restricted by changing the effective diameter and surface area of each lumen.

In one embodiment, at least one flow regulator 214 may comprise at least one valve configured to provide a variable flow coefficient when positioned within the lumen. In one embodiment, the valve may operate with any mechanism known to one skilled in the art including but not limited to a sliding action valve, gate valve, etc. In one embodiment, the valve may comprise an internal flapper wherein the angle of the flapper changes the effective flow/pressure rate of the lumen.

In one embodiment, at least one sensor 212 is positioned at distal end 206 with at least one flow regulator 214 positioned at proximal end 204. In one embodiment, at least one sensor 212 and at least one flow regulator 214 are positioned at proximal end 204 (FIG. 20).

At least one sensor 212 and at least one flow regulator 214 may be communicatively connected to a processor. The processor is configured to perform computing steps, including sensor reading steps for example obtaining one or more samples from at least one sensor 212, and/or parameter adjustment steps, comprising sending data to at least one sensor 212 or at least one flow regulator to activate/deactivate the sensors or adjust flow in each lumen. In some embodiments, the processor may include actuation steps performed in response to particular values of at least one sensor 212 measurement, for example activating at least one flow regulator 214 to adjust flow/and or pressure in first lumen 203 and/or second lumen 205.

Insertion and placement of device 200 may be achieved using any desired method. In one embodiment, device 200 may be inserted or retracted over a guidewire or a stylet. In one embodiment, the stylet/guidewire may have a flexible shaft configured to withstand and transmit torque as applied by the user. In one embodiment, the stylet/guidewire may comprise a tip that may be directed by the user. In one embodiment, the stylet/guidewire may further comprise a camera device positioned at the tip to allow the user to view the patient's anatomy during the intubation process. In one embodiment, the stylet/guidewire can be routed through any of the lumens of the device 200.

In one embodiment, device 200 may be used in a variety of settings including but not limited to an in-patient setting where a patient is mechanically ventilated and intubated.

### Method of Use

The present invention relates to methods of regulating gas exchange in the lungs that is capable of delivering variable flow and pressure rates of gas to the left and right lungs independently. The regulation of flow is adjustable by the operator and is based on quantified parameters of physiologic or pathologic differences between the left and right lung represented by dynamic and static direct monitoring of pressures, EtCO₂, or other physiologic or pathologic parameters in each lung independently. In one embodiment, method of the present invention allows for asymmetric ventilation to left and right lung based on these parameters, as well as quantification of the pathophysiological differences between the two lungs, allowing for improved patient care and reduced injury. In one embodiment, the method of the present invention may be used for patients with unilateral, asymmetric, or heterogenous lung injury requiring mechanical ventilation.

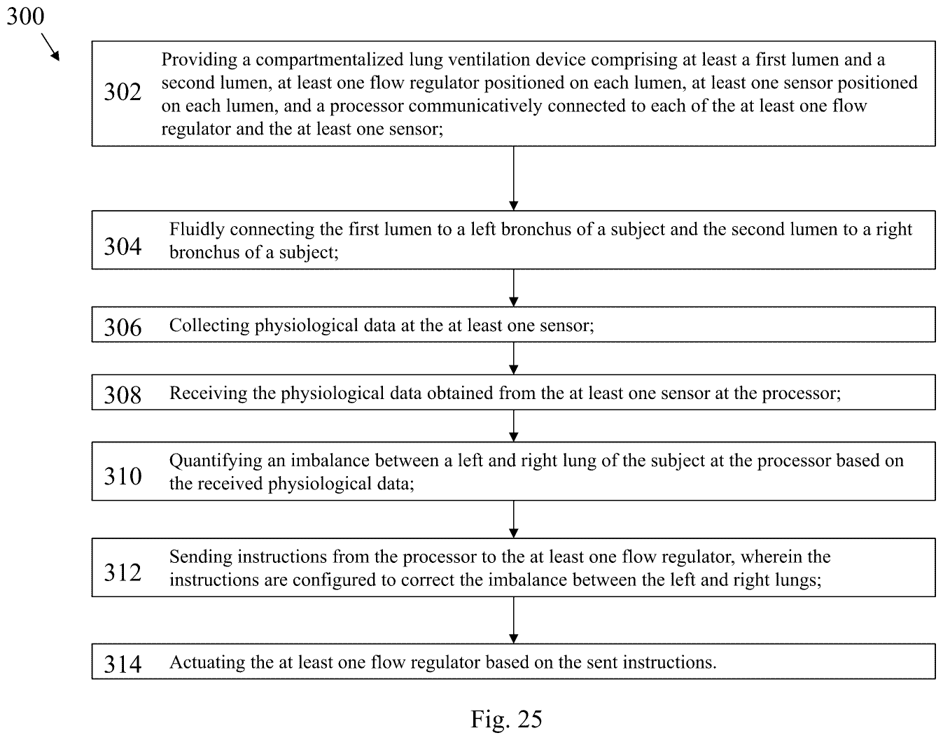

Referring now to FIG. 25, an exemplary method of regulating gas exchange in the left and right lungs independently using device 100 and 200 is depicted. Method 300 begins with step 302 wherein a compartmentalized lung ventilation device comprising at least a first lumen and a second lumen, at least one flow regulator positioned on each lumen, at least one sensor positioned on each lumen, and a processor communicatively connected to each of the at least one flow regulator and the at least one sensor is provided. In step 304, the first lumen is fluidly connected to a left bronchus of a subject and the second lumen is fluidly connected to a right bronchus of a subject. In step 306, physiological data is collected at the at least one sensor. In step 308, the physiological data obtained from the at least one sensor is received at the processor. In step 310, a variation between a left and right lung of the subject is quantified at the processor based on the received physiological data. In Step 312, instructions are sent from the processor to the at least one flow regulator, wherein the instructions are configured to correct the variation between the left and right lungs. In step 314, the at least one flow regulator is actuated based on the sent instructions.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### Example 1

As there is a lack of a standard lung model which can independently measure plateau pressure in each lung, a novel simulation model was developed in which either of two high-fidelity breathing simulators act as a single lung. The breathing simulators (ASL 5000 Breathing Simulators, IngMar Medical, Pittsburgh, PA) used in this study can provide a wide range of compliance and resistance values. Compliance ranges from 0.5 to 250 milliliters per centimeters of water (mL/cm H2O) and resistance ranges from 3 to 500 centimeters of water per liter per second (cm H2O/L/s). Calibration of breathing simulators was performed by the manufacturer per their recommendations.

The materials and methods employed in these experiments are now described.

### Ventilatory Methods

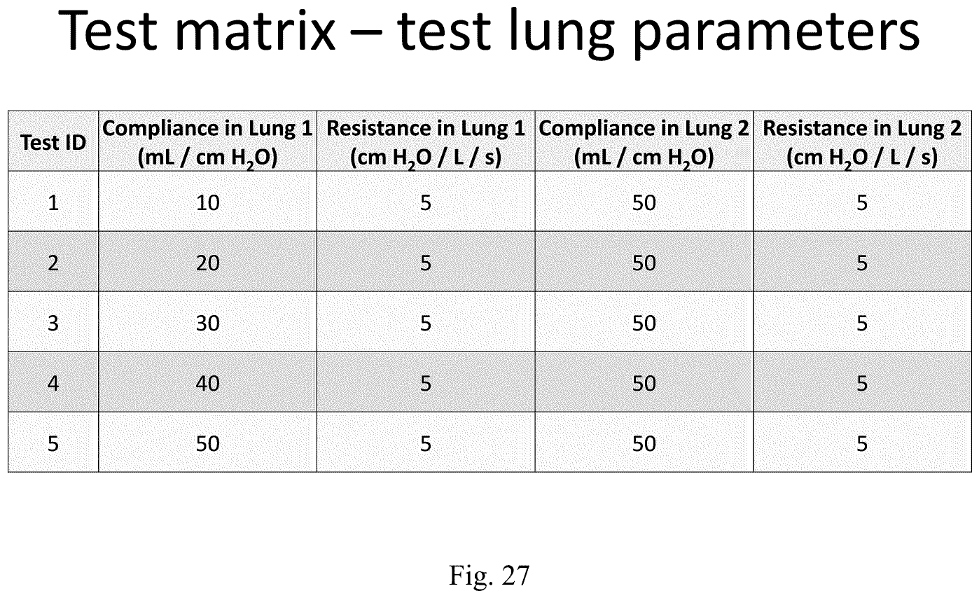

A range of test cases was evaluated, with varying compliance and resistance applied to the breathing simulators, labeled below as lung 1 (L1) and lung 2 (L2). The compliance and resistance values in L2 (Cu and Ru, respectively) remained constant at 50 mL/cm H2O and 5 cm H2O/L/s, respectively. The compliance and resistance values in L1 (C_{L1} and R_{L1}, respectively) are varied to represent a wide array of lung asymmetries and clinical pathologies (FIG. 27).

Each test case is run for a pre-determined number of cycles. There is no PEEP value target—the system intrinsic PEEP, which depends on baseline characteristics and lung resistance, determines the pressure at the end of expiration. Following the end of the inspiratory phase, a compartmentalized inspiratory hold is performed to independently determine the static, plateau pressure in L1 and L2 (P_{plat,L1} and P_{plat,L2}) by using the isolation valves to prevent equilibration between the two lungs. After the compartmentalized inspiratory hold is complete, a standard inspiratory hold is performed where system plateau pressure (P_{plat,SYS})is determined. Data were also collected on the peak pressures in L1 and L2 (P_{peak,L1} and P_{peak,L2}, respectively). Tidal volumes were measured in L1, L2 and the system (V_{T,L1}, V_{T,L2}, and V_{T,SYS}, respectively).

At the end of the ventilatory cycle, a short expiratory hold is performed, allowing pressure in the lung simulator, tubing, and ventilator circuit to equilibrate between cycles. This cycle is repeated for the duration of the test.

### Data Collection

All data were collected using ASL 5000 Software version 3.6. This collects data on several different channels from internal device sensors at 512 Hz. The data were analyzed using Python scripts. Flow, pressure, and volume data are extracted from the breathing simulators. Verification of static compliance and resistance of each test case is performed with the data collected by the breathing simulators.

A script was used to ensure proper time synchronization between the two breathing simulators. The analysis scripts align the data for each test case based on the pressure sensor readings from the cycles between the two devices.

### Data Analysis

Analyses were performed to understand the variations that exist between L1 and L2 across all test cases. Parameters that were analyzed include V_{T,L1}, V_{T,L2}, V_{T,SYS}, P_{peak,L1}, P_{peak,L2}, P_{plat,L1}, P_{plat,L2}, and P_{plat,SYS} across different test cases.

Ratios of each parameter between L1 and L2 were calculated. Analyses determined the ratio of V_{T,L1}/V_{T,L2}; the ratio of P_{peak,L1}/P_{peak,L2}; and the ratio of P_{plat,L1}/P_{plat,L2}.

The results of these experiments are now described.

### Results

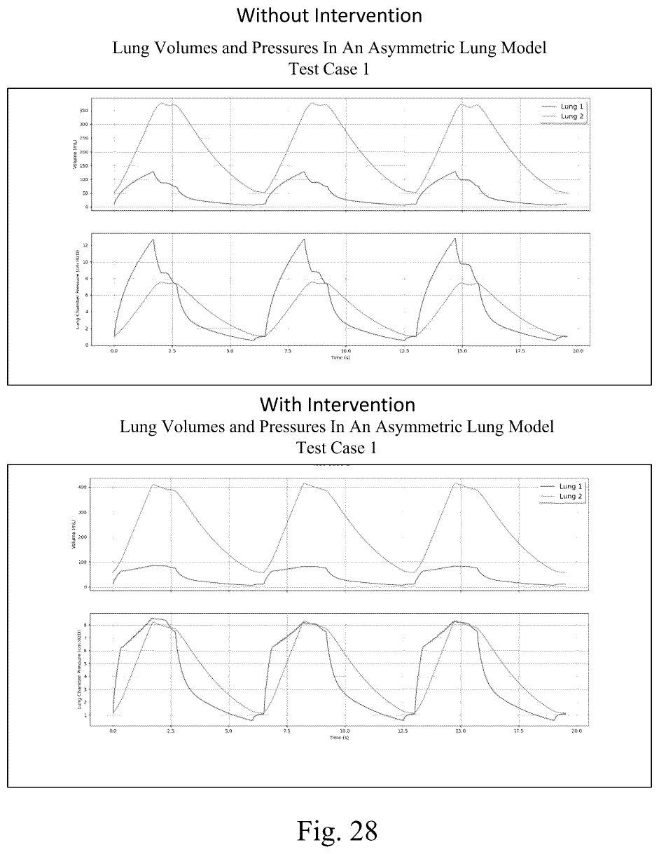

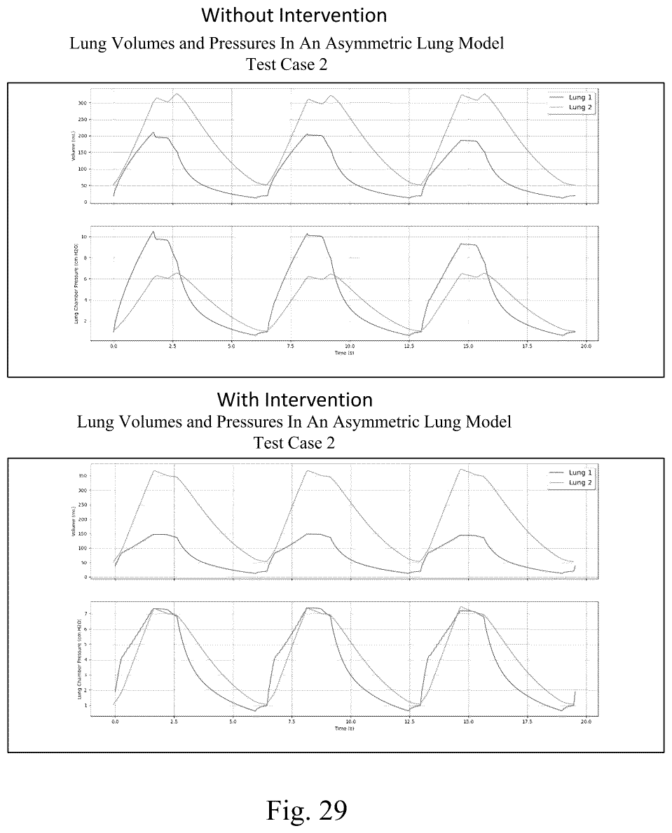

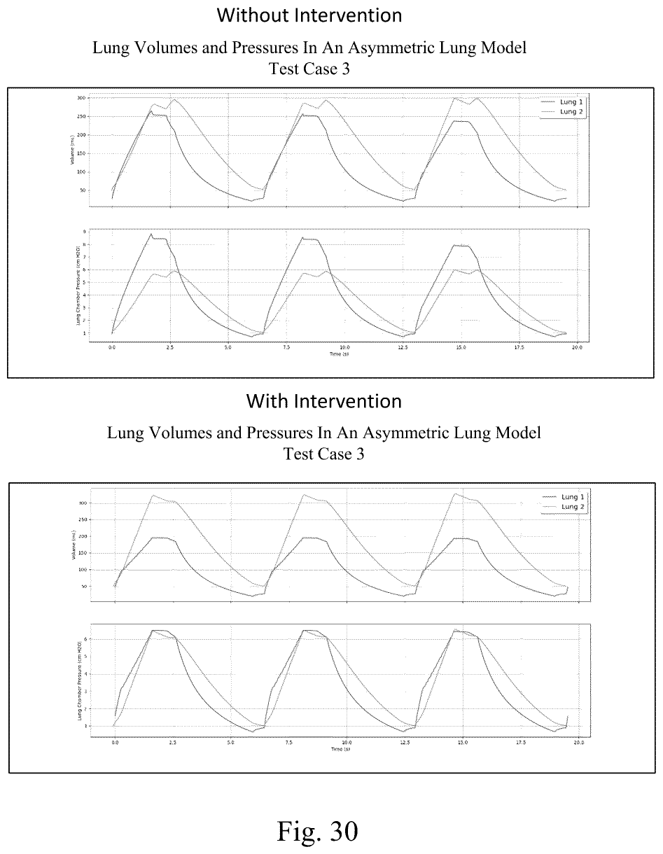

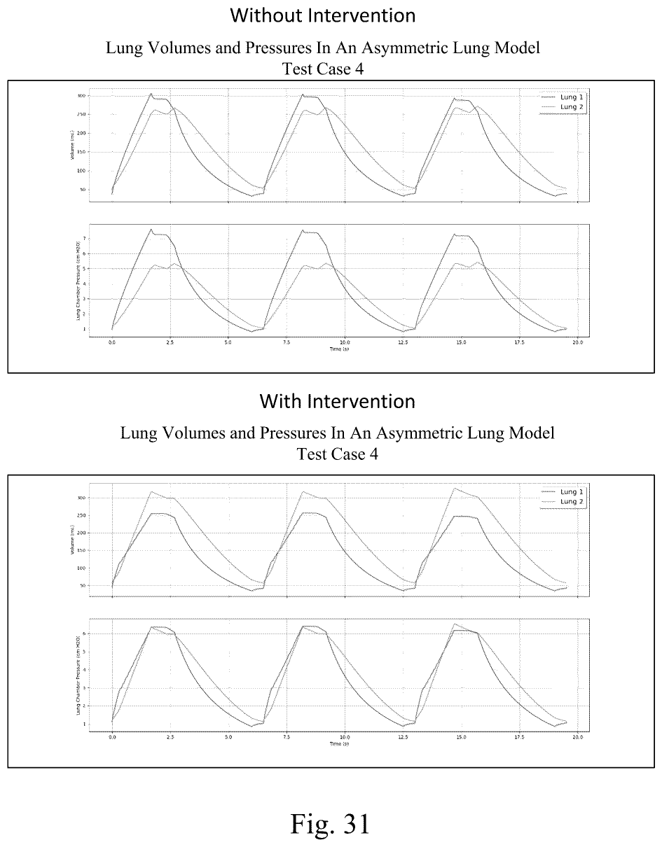

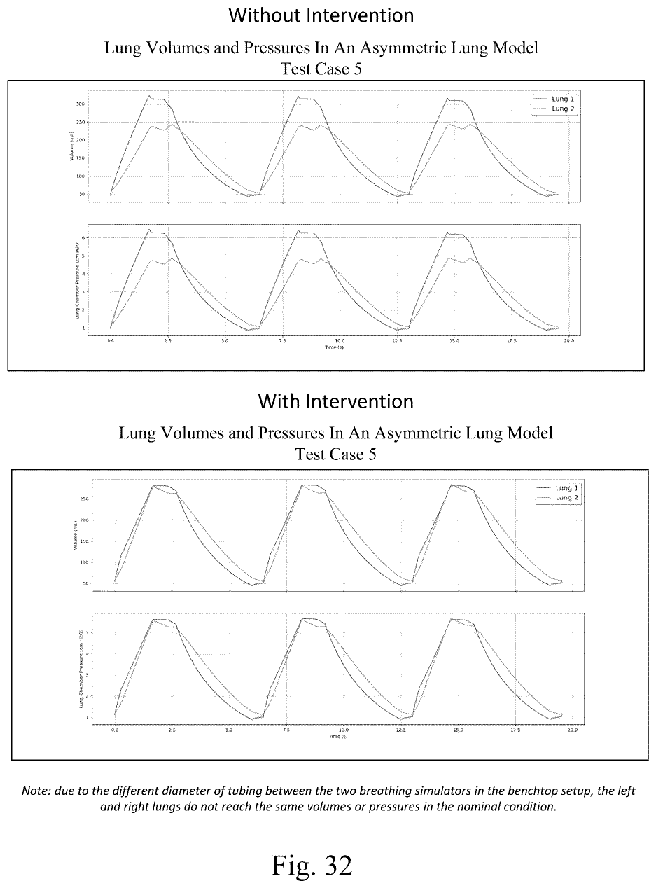

The results of test cases 1-5 are shown in FIG. 28 through FIG. 32. Here, volume and pressure waveforms show the impacts of maldistribution of gas volume between an injured and normal lung in an asymmetric lung injury model. Without the use and intervention of the compartmentalized lung ventilation device of the present invention, significant asymmetries in gas pressure were present between the two lungs.

With the use of the compartmentalized lung ventilation device of the present invention, pressure is equalized between the two lungs, which results in an alteration of the volumetric distribution of gas in a non-pathophysiologic way.

Without any intervention, the distribution of gas between the two lungs was asymmetric with only about 29% of the total volume going to the injured lung. Despite this, the high compliance variation meant that the injured lung still reached a higher peak pressure than the normal lung, with intra-lung pressures up to 84% higher than in the normal lung.

After intervention, the distribution of gas became even more asymmetric. This is required as lower volumes must be delivered to the injured lung in order to decrease high pressures. After intervention, as little as 16% of the total tidal volume was delivered to the injured lung, with the remaining volume going to the normal lung. This resulted in approximately even pressures between the two lungs.

Different test cases involve varying parameters of the test lungs and thus different volume and pressure distributions of gas between the two lungs.

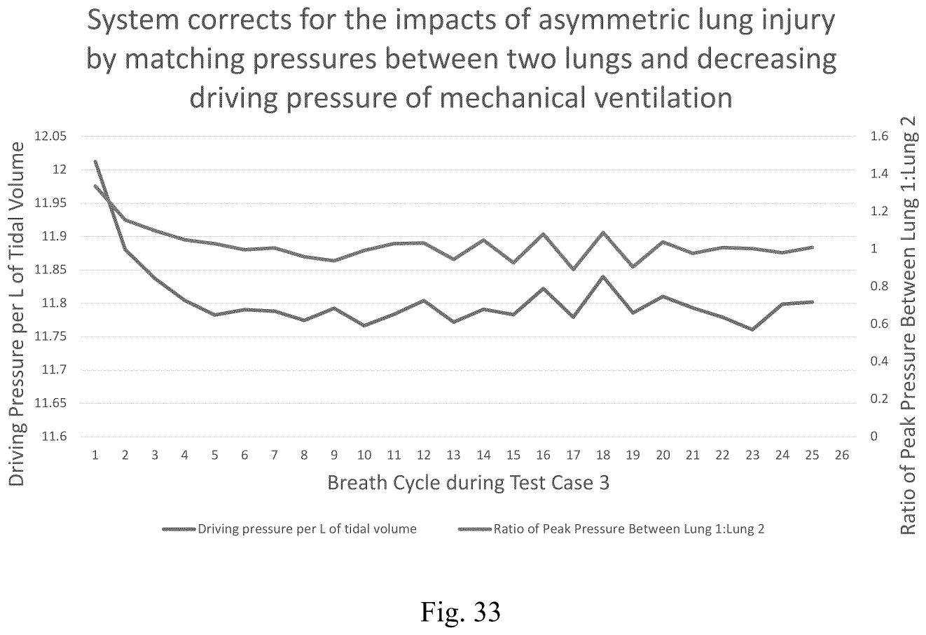

Using a novel asymmetric lung injury model of the present invention, it was shown that there is a significant maldistribution of delivered gas and static and dynamic pressures coming as a result of asymmetric lung compliance and inspiratory airflow resistance. By implementing a closed loop control algorithm that adjusts the volumetric distribution of gas between the two lungs to equalize lung pressure, a reduction in the driving pressure of ventilation in cases of severe asymmetry was demonstrated (FIG. 33).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A compartmentalized lung ventilation device, comprising:
a first lumen having a proximal end and a distal end;
a second lumen;
a third lumen;
at least one flow regulation mechanism positioned along the length of each lumen;
at least one port positioned along the length of the second lumen and/or the third lumen, comprising at least one sensor; and
a processor;
wherein the first lumen is fluidly connected to a mechanical ventilator circuit at the proximal end and is fluidly connected to the second lumen and the third lumen at the distal end; and wherein the second lumen is distally connected to left mainstem bronchus, and the third lumen is distally connected to right mainstem bronchus of a subject.

2. The compartmentalized lung ventilation device of claim 1, wherein the at least one sensor is selected from the group consisting of a pressure sensor, an end-tidal carbon dioxide (EtCO₂) sensor, an end-tidal oxygen (EtO₂) sensor, a flow sensor, a gas concentration sensor, and combinations thereof, and wherein the at least one sensor is configured to quantify regional variations in pathophysiology between regions of a subject's lung.

3. The compartmentalized lung ventilation device of claim 1, wherein the at least one flow regulator modulates the flow to the left mainstem bronchus and the right mainstem bronchus via at least one mechanism selected from the group consisting of: an inflatable member, an internal flapper, a pinch mechanism, and a valve mechanism to provide a variable flow coefficient to the tube.

4. The compartmentalized lung ventilation device of claim 3, wherein the inflatable member is positioned within the wall of each of the second lumen and the third lumen.

5. The compartmentalized lung ventilation device of claim 3, wherein the inflatable member is inflated or deflated by providing a precisely measured volume of fluid to the inflatable member, wherein the fluid is introduced by one selected from the group consisting of: manual application using a syringe, an automated control box with software modules that control the level of inflation and deflation based on at least one sensor measurements.

6. The compartmentalized lung ventilation device of claim 3, wherein the internal flapper is configured to change the effective volume of gas, flow, and/or pressure rate in each of the first lumen, second lumen and the third lumen by changing the angle of the flapper.

7. The compartmentalized lung ventilation device of claim 3, wherein the inflatable member may be either inflated or deflated by providing a precisely measured volume of fluid to the inflatable member, wherein the fluid is introduced by one selected from the group consisting of: manually using a syringe, an automated control box with software modules that control the level of inflation and deflation based on compartmentalized sensor measurements.

8. The compartmentalized lung ventilation device of claim 3, wherein the pinch mechanism comprises a pinch arm positioned externally around the second lumen and the third lumen and is configured to allow compression of each lumen to change the effective diameter and surface area of each lumen.

9. The compartmentalized lung ventilation device of claim 3, wherein the valve mechanism may be one selected from the group consisting of: a pinch valve, a ball valve, a butterfly valve, a needle valve, a solenoid valve, a sliding action valve, and a gate valve.

10. The compartmentalized lung ventilation device of claim 1, wherein the first lumen is fluidly connected to the mechanical ventilator circuit at the proximal end through a tubing adapter.

11. The compartmentalized lung ventilation device of claim 1, wherein the processor further comprises a software platform comprising a regulation control module (RCM), a clinical parameter module (CPM) and an alarm module (AM), wherein at least one of the RCM, CPM and AM is configured to regulate flow through each of the first lumen, second lumen and the third lumen based on a signal received from the at least one sensor.

12. The compartmentalized lung ventilation device of claim 1, wherein the processor further comprises a software platform having a closed loop controller, wherein the closed loop controller is configured to use a proportional-integral-derivative controller (PID controller) for achieving a correct flow rate of gas through the second lumen and/or the third lumen.

13. The compartmentalized lung ventilation device of claim 12, wherein the closed loop controller comprises: a clinical parameter configured to adjust peak pressure in left and right lungs in a non-pathophysiologic manner; two pressure sensors positioned at the distal end of the second lumen and/or the third lumen; an error term in the PID controller which is the difference between the measured pressure in the left and right lungs and the target differential between the lungs; and a closed-loop control module configured to modulate the at least one flow regulator to minimize the error.

14. The compartmentalized lung ventilation device of claim 12, wherein the closed loop controller is configured to use inputs from the at least one sensor and a desired clinical parameter to calculate an error, based on regional variations and desired clinical outcome for the subject.

15. The compartmentalized lung ventilation device of claim 1, wherein the processor further comprises advanced algorithms, selected from the group consisting of a machine learning algorithm based on supervised learning and unsupervised learning, wherein in supervised machine learning, data from the device is compared to traditional lung performance test results and other clinical tests and wherein in unsupervised machine learning, time-based data from the device is used to develop a model based on how future lung performance is impacted by past lung performance.

16. The compartmentalized lung ventilation device of claim 15, wherein the advanced algorithm is used to provide data-informed care, predictive care, personalized medicine and improve the subject's outcomes.

17. The compartmentalized lung ventilation device of claim 15, wherein the processor is configured to use a compartmentalized inspiratory hold to measure a clinical parameter in a compartmentalized no-flow condition.

18. The compartmentalized lung ventilation device of claim 1, wherein the device is used in an in-patient setting.

19. The compartmentalized lung ventilation device of claim 1, wherein the at least one port is positioned distal to the at least one flow regulator.

20. The compartmentalized lung ventilation device of claim 1, wherein the at least one port comprises a cap to prevent leakage of gas from a subject's circuit.

21. The compartmentalized lung ventilation device of claim 1, wherein the at least one port comprises a tubing adapter, configured to allow easy attachment to external devices.

22. The compartmentalized lung ventilation device of claim 1, wherein the at least one flow regulator is configured to regulate a flow of gas between various regions of a subject's lungs by at least one mechanism selected from the group consisting of equalizing the pressure or EtCO₂ between the regions of the lungs, or achieving an unequal, but different from baseline, distribution of pressure or EtCO₂ between regions of the lungs.

23. The compartmentalized lung ventilation device of claim 1, further comprising an imaging system selected from the group consisting of: an x-ray and a computed tomography (CT) scan configured to collect data on variations in regional pathophysiology of the lungs.

24. A method of regulating gas exchange in the left and right lungs independently comprising the steps of:
providing a compartmentalized lung ventilation device comprising at least a first lumen and a second lumen, at least one flow regulator positioned on each lumen, at least one sensor positioned on each lumen, and a processor communicatively connected to each of the at least one flow regulator and the at least one sensor;
fluidly connecting the first lumen with a left bronchus of a subject and the second lumen with a right bronchus of a subject;
collecting physiological data at the at least one sensor;
receiving the physiological data obtained from the at least one sensor at the processor;
quantifying a variation between a left and right lung of the subject at the processor based on the received physiological data;
sending instructions from the processor to the at least one flow regulator, wherein the instructions are configured to correct the variation between the left and right lungs; and
actuating the at least one flow regulator based on the sent instructions.

25. The method of claim 24, wherein the at least one sensor is selected from the group consisting of a pressure sensor, an EtCO₂ sensor, an EtO₂ sensor, a flow sensor, a gas concentration sensor, and combinations thereof.

26. The method of claim 24, wherein the at least one flow regulator modulates the flow to the left mainstem bronchus and the right mainstem bronchus via at least one mechanism selected from the group consisting of: an inflatable member, an internal flapper, a pinch mechanism, and a valve mechanism to provide a variable flow coefficient to the tube.

27. The method of claim 26, wherein the inflatable member is positioned within the wall of each of the first lumen and second lumen.

28. The method of claim 26, wherein the internal flapper is configured to changes the effective flow and/or pressure rate in each of the first lumen, second lumen and the third lumen by changing the angle of the flapper.

29. The method of claim 26, wherein the pinch mechanism comprises a pinch arm positioned externally around each of the first lumen, second lumen and the third lumen and is configured to allow compression of each lumen to change the effective diameter and surface area of each lumen.

30. The method of claim 26, wherein the valve mechanism may be one selected from the group consisting of: a pinch valve, ball valve, butterfly valve, needle valve, solenoid valve, sliding action valve, gate valve.
